(19) United States
(12) Patent Application Publication
(10) Pub. No.: US 2024/0384346 A1
(43) Pub. Date: Nov. 21, 2024

(54) ORAL SWAB-BASED TEST FOR THE DETECTION OF DENTAL DISEASE STATES IN DOMESTIC CATS, DOGS AND OTHER MAMMALS

(71) Applicant: Basepaws, Torrance, CA (US)

(72) Inventors: YULIANA MIHAYLOVA, PLAYA VISTA, CA (US); DAMIAN KAO, PLAYA VISTA, CA (US)

(21) Appl. No.: 18/556,044

(22) PCT Filed: Apr. 22, 2022
(86) PCT No.: PCT/US2022/071852

§ 371 Date: Oct. 18, 2023

Related U.S. Application Data
(60) Provisional application No. 63/178,395, filed on Apr. 22, 2021.
(60) Provisional application No. 63/221,554, filed on Jul. 14, 2021.

(51) Int. Cl. C12Q 1/6883; C12Q 1/689; G16H 50/30; G16H 50/70

(52) U.S. Cl. CPC C12Q 1/6883; C12Q 1/689; G16H 50/30; G16H 50/70; C12Q 2600/118; C12Q 2600/158

(57) ABSTRACT

Systems and methods for screening for and identifying oral disease states in domestic cats, dogs and other mammals.

### CROSS-REFERENCE TO RELATED APPLICATIONS

This application claims the benefit of and priority to: (1) U.S. Provisional Application No. 63/178,395, filed Apr. 22, 2021, titled “Development of an Oral Swab Based Microbiome Test for the Detection of Feline Dental Disease,” and (2) U.S. Provisional Application No. 63/221,554, filed Jul. 14, 2021, titled “Oral Swab-Based Test for the Detection of Dental Disease States in Domestic Cats, Dogs, and Other Mammals,” the entirety of each of which is incorporated herein by specific reference.

### BACKGROUND

### Technical Field

This disclosure relates to systems and methods for screening for, detecting and identifying oral disease states in domestic cats, dogs and other mammals.

### Related Technology

Dental health in cats and dogs, and mammals, in general, is known to be linked to the overall health and wellbeing of the individual animal. That is, dental health may be a good proxy for overall health and wellbeing in cats, dogs and mammals more generally. Dental conditions may be indicative of wider, more serious systemic conditions and may impact an individual animal's level of comfort while living with a particular dental condition. For example, animals suffering from dental disease conditions may experience pain, loss of sleep, loss of appetite, decreased activity, and depression, among other things.

One of the most prevalent forms of mammalian dental disease, periodontal disease, can generally be broken down into four stages, where the gingiva (gums) becomes inflamed in stage 1. In stages 2-4, varying degrees of tooth support are lost until, in stage 4, over 50% of the tooth support is lost. This can result in loss of teeth for the mammal and pain when using the teeth (such as during eating). Many mammalian animals, such as cats and dogs, are incapable of communicating this pain and discomfort to their owners. Moreover, by the time dental disease-associated pain begins to manifest, it is too late for prevention focused regimens to significantly improve oral health and some treatment options may be unavailable or ineffective, resulting in increased owner spending for emergency veterinary services.

Many mammalian animals, such as cats and dogs, do not receive routine veterinary care, meaning that early signs of dental disease can often be missed. To compound this problem, extensive assessment of oral health is rarely a part of any routine veterinary visit. The typical veterinary oral health examination relies on a visual inspection of the mouth during an awake exam. Ensuring that early signs of developing dental disease are not missed during an examination requires the animal to be put under anesthesia and undergo X-ray imaging of the oral cavity, since signs of dental disease are often invisible to the naked eye. Due to the high cost of this procedure and the risks associated with cats and dogs undergoing anesthesia, this is not standard practice in most veterinary hospitals and clinics.

Accordingly, there is a need for robust and accurate, yet safe, painless and affordable means that can be used on a recurring basis for detecting dental disease in mammalian animals, such as cats and dogs. Using such a tool to guide and complement veterinary oral health assessment can significantly improve oral health outcomes and lead to detecting signs of deteriorating oral health and implementing treatment earlier compared to relying on veterinary visits alone.

### SUMMARY

Embodiments of the present disclosure include systems and methods for screening for, detecting and identifying oral disease states in cats, dogs and/or other mammalian companion animals. Embodiments of the disclosed subject matter describe a method for interrogating the oral microbiome of a mammalian companion animal. The disclosed methods interrogate the oral microbiome to detect microbe compositional abundance trends that may be associated with dental disease in cats, dogs and other mammalian animals. Detecting, identifying and/or quantifying microbial compositional abundance trends enables a practitioner to screen for and/or indicate whether a cat, dog and/or other mammal has a particular oral and/or dental disease state. Detecting and identifying oral and/or dental disease states enables the practitioner and/or the mammalian animal's owner to treat and/or prevent the dental disease state. Treating and/or preventing oral disease states enables treatment and/or prevention of wider, systemic conditions, beneficially resulting in a healthier and more comfortable life for the mammalian animal.

In some embodiments, a method is disclosed for detecting and/or indicating oral disease in mammalian animals. The method may include receiving an oral swab sample taken from a mammalian animal; manipulating the sample, such as heat treatment of the oral sample; and extracting microbial deoxyribonucleic acids (DNA) from the heat-treated sample. The method may additionally include sequencing the microbial DNA to identify which specific one or more microbes are present in the oral sample (and in what relative proportions), wherein identifying the specific one or more microbes enables generation of an oral microbial profile for the mammalian animal. The method may additionally include comparing the oral microbial profile for the mammalian animal against a reference database including defined microbial profiles, wherein the database identifies correlations between (i) profiles that include one or more microbes and (ii) corresponding oral diseases; and based on a result of comparing the oral microbial profile against the database of defined microbial profiles, generating a risk score indicating a likelihood that the mammalian animal has a specific oral disease.

The method may further include treating the specific oral disease and/or administering a therapeutic treatment. In some embodiments, the therapeutic treatment may include administering a therapeutic compound, such as a compound designed to inhibit or encourage growth of a specific one or more microbes present in the oral microbiome of the mammal. In some embodiments, the therapeutic compound includes a pre-biotic, a post-biotic, a pro-biotic, a medicament or a combination thereof. In some embodiments, the therapeutic treatment may include brushing the mammal's teeth with a topical treatment.

In some embodiments, a method for indicating oral disease in mammalian animals includes receiving an oral swab sample taken from a mammalian animal and performing heat treatment on the oral sample. The method may also include performing magnetic beads-based deoxyribonucleic acid (DNA) extraction on the heat treated oral sample to extract microbial DNA that is present in the oral swab sample and sequencing the microbial DNA to identify which specific one or more microbes are present in the oral sample (and in what compositional abundance), wherein identifying the specific one or more microbe(s) enables generation of an oral microbial profile for the mammalian animal. The method may additionally include comparing the oral microbial profile for the mammalian animal against a database of defined microbial profiles, wherein the database identifies correlations between (i) profiles that include one or more microbes (and their compositional abundance) and (ii) corresponding oral diseases; and based on a result of comparing the oral microbial profile against the database of defined microbial profiles, generating a risk score indicating a likelihood that the mammalian animal has a specific oral disease. The method may include, in response to generating the risk score and identifying the specific oral disease, administering a therapeutic treatment designed to treat the specific oral disease, recommending veterinary attention or follow-up examination, and/or recommending at-home care for specific oral diseases.

Also disclosed are computer systems. In some embodiments, a computer system is configured to indicate oral disease in mammalian animals and includes one or more processors and one or more computer-readable hardware storage devices that store instructions executable by the one or more processors. The instructions may configure the computer system to receive sequenced microbial DNA data from an oral swab sample taken from a mammalian animal; map the sequenced microbial DNA to identify which specific one or more microbial species are present in the oral sample, wherein identifying the specific one or more microbial species results in generation of an oral microbial profile for the mammalian animal; calculate relative abundance of different microbial species to further build the oral microbial profile for the mammalian animal; compare the oral microbial profile for the mammalian animal against a database of defined microbial profiles, wherein the database identifies correlations between (i) profiles that include one or more microbial species and their relative abundance and (ii) corresponding oral diseases; and based on a result of comparing the oral microbial profile against the database of defined microbial profiles, generate a risk score indicating a likelihood that the mammalian animal has a specific oral disease. In response to generating the risk score, the instructions may further configure the computer system to generate a report outlining and/or presenting the risk score and prescribing a therapeutic treatment and/or at-home treatment protocol suitable for addressing (e.g., treating and/or preventing) the specific oral disease. The therapeutic treatment protocol may be influenced by the severity of the oral disease state, which is indicated by or correlated to the risk score.

In some embodiments, the therapeutic treatment or at-home care protocol is designed to alter the composition of the oral microbiome of the mammalian animal. In some embodiments, altering the composition of the mammalian animal's oral microbiome treats and/or addresses the specific oral disease. In some embodiments, the therapeutic treatment repairs the mammalian animal's oral microbiome. In some embodiments, the therapeutic treatment or at-home care protocol is designed to maintain the composition of the oral microbiome of the mammalian animal. In some embodiments, the therapeutic treatment protocol is designed to stimulate a metabolic output of the mammalian animal's oral microbiome. Stimulating a metabolic output of the mammalian animal's oral microbiome may include using known enzymatic pathway analysis tools to provide an additional dimension to the existing microbial composition data to further characterize disease signatures and improve predictive disease models.

Illustrative embodiments and non-limiting examples of the present disclosure include:
Example 1. A method for screening for, detecting, and/or preventing oral disease in non-human, mammalian animals, the method comprising:
   obtaining an oral microbial profile for a non-human, mammalian animal, the oral microbial profile comprising one or more microbial species present in an oral sample of the non-human, mammalian animal and a quantity or abundance of the one or more microbial species in the oral sample;
   comparing the oral microbial profile to information in a database that identifies weighted correlations between:
   (i) occurrence and/or prevalence of one or more oral diseases in animals in a classification of the non-human, mammalian animal; and
   (ii) presence and/or abundance of various microbial species in the oral microbiome of animals in the classification of the non-human, mammalian animal, wherein the various microbial species comprise the one or more microbial species in the oral sample;
   generating a risk score indicating a likelihood that the non-human, mammalian animal has the one or more oral diseases based on one or more matches between the oral microbial profile and the information in the database; and
   categorizing the non-human, mammalian animal as developing the one or more oral diseases when the risk score meets or exceeds a predetermined threshold and, optionally, prescribing a therapeutic treatment protocol suitable for treating, mitigating, or preventing the development, advancement, or recurrence of the one or more oral diseases when the risk score meets or exceeds a predetermined threshold.
Example 2. The method of Example 1 further comprising administering the therapeutic treatment protocol to the non-human, mammalian animal or confirming that the therapeutic treatment protocol has been administered to the non-human, mammalian animal, wherein the therapeutic treatment protocol is sufficient to alter the oral microbial profile of the non-human, mammalian animal.
Example 3. The method of Example 1, wherein obtaining the oral microbial profile for the non-human, mammalian animal comprises:
   obtaining nucleic acid sequence data corresponding to microbial nucleic acid obtained from the oral sample;
   analyzing the nucleic acid sequence data to identify the one or more microbial species present in the oral sample and quantifying the one or more microbial species; and
   generating the oral microbial profile for the non-human, mammalian animal based on the identified and, optionally, quantified one or more microbial species.
Example 4. The method of Example 3, wherein obtaining the microbial nucleic acid sequence data comprises:
   sequencing microbial nucleic acid from the oral sample; and, optionally,
   isolating the microbial nucleic acid from the oral sample.
Example 5. The method of Example 4, wherein isolating the microbial nucleic acid from the oral sample comprises:
   performing heat treatment on the oral sample; and
   performing magnetic SPRI beads-based nucleic acid extraction on the heat-treated oral sample, with or without the addition of protein digesting reagents and detergents, to extract the microbial nucleic acid from the oral sample.
Example 6. The method of Example 3, wherein analyzing the microbial nucleic acid sequence data comprises one or more of:
   demultiplexing the nucleic acid sequence data;
   trimming the nucleic acid sequence data;
   mapping one or more unmapped reads onto a reference genome of the non-human, mammalian animal and/or onto existing microbial reference genomes;
   classifying one or more reads as mammalian from the nucleic acid sequence data after mapping;
   classifying one or more reads as microbial from the nucleic acid sequence data after mapping;
   quantifying the one or more microbial reads;
   transforming the quantified one or more microbial reads to account for sequence coverage biases using methods such as pairwise log ratio transformation; and
   comparing compositional abundance patterns in the transformed one or more microbial reads against compositional abundance patterns in the transformed data in a reference database comprising samples from non-human, mammalian animals that do not suffer from dental diseases, as well as samples from non-human, mammalian animals that suffer from specific dental diseases.
Example 7. The method of Example 1, wherein comparing the oral microbial profile to the information in the database comprises one or more of:
   calculating the abundance of the one or more microbial species in the oral sample;
   identifying the one or more microbial species in the oral sample; and
   comparing the abundance of the identified one or more microbial species in the oral sample to the presence and/or abundance of various microbial species in the oral microbiome of animals in the classification of the non-human, mammalian animal contained in the database.
Example 8. The method of Example 1, wherein generating the risk score comprises one or more of:
   identifying one or more similarities between compositional abundance of the one or more microbial species in the oral sample and compositional abundance of various microbial species in the oral microbiome of animals in the classification of the non-human, mammalian animal contained in the database;
   identifying one or more matches between the identity of the one or more microbial species in the oral sample and the presence of various microbial species in the oral microbiome of animals in the classification of the non-human, mammalian animal contained in the database;
   quantifying the identified one or more similarities between the compositional abundance of the one or more microbial species in the oral sample and the compositional abundance of the one or more microbial species in the oral microbiome of animals in the classification of the non-human, mammalian animal contained in the database; and
   identifying a presence of one or more predictive microbial species in the oral sample.
Example 9. The method of Example 1, wherein the one or more oral diseases is selected from the group consisting of periodontal disease, tooth resorption, gingivostomatitis, and halitosis.
Example 10. The method of Example 1 further comprising:
   generating a report presenting (i) the risk score, (ii) an indication of developing the one or more oral diseases when the risk score meets or exceeds the predetermined threshold, (iii) a timing recommendation, (iv) optionally, one or more at home practices to improve dental health, (v) optionally, one or more diagnostic steps to diagnose the one or more oral diseases when the risk score meets or exceeds the predetermined threshold, and (vi) optionally, a prescription for the therapeutic treatment protocol; and, optionally,
   communicating the generated report electronically to an owner of the non-human, mammalian animal and/or their veterinarian.
Example 11. The method of Example 1, wherein the therapeutic treatment protocol is sufficient to alter the oral microbial profile of the non-human, mammalian animal.
Example 12. A computer system configured to indicate or predict oral disease in mammalian animals, the computer system comprising:
   one or more processors; and
   one or more computer-readable hardware storage devices having stored thereon instructions that are executable by the one or more processors to configure the computer system to:
   receive microbial nucleic acid sequence data corresponding to microbial nucleic acid obtained from an oral sample taken from a mammalian animal;
   analyze the microbial nucleic acid sequence data to identify one or more microbial species present in the oral sample and quantify the one or more microbial species;
   generate an oral microbial profile for the mammalian animal based on the identified one or more microbial species and their respective abundances;
   compare the oral microbial profile to information in a database that identifies weighted correlations between:
   (i) occurrence and/or prevalence of one or more oral diseases in animals in a classification of the mammalian animal; and
   (ii) presence and/or abundance of various microbial species in the oral microbiome of animals in the classification of the mammalian animal, wherein the various microbial species comprise the one or more microbial species in the oral sample;
   identify one or more matches between the oral microbial profile and the information in the database;
   generate a risk score indicating a likelihood that the mammalian animal has the one or more oral diseases based on the one or more matches between the oral microbial profile and the information in the database; and, optionally,
   diagnose the mammalian animal as “developing” the one or more oral diseases when the risk score meets or exceeds a predetermined threshold,
   prescribe a therapeutic treatment protocol suitable for treating or preventing the one or more oral diseases when the risk score meets or exceeds the predetermined threshold,
   generate a report indicating (i) the risk score, (ii) an indication of developing the one or more oral diseases when the risk score meets or exceeds the predetermined threshold, (iii) a timing recommendation, (iv) optionally, one or more at home practices to improve dental health, (v) optionally, one or more diagnostic steps to diagnose the one or more oral diseases when the risk score meets or exceeds the predetermined threshold, and (vi) a prescription for the therapeutic treatment protocol, and/or
   communicate the generated report electronically to an owner of the mammalian animal and/or their veterinarian.
Example 13. The computer system of Example 12, wherein the instructions further configure the computer system to map one or more unmapped reads to a mammalian reference genome and/or map one or more reads to microbial reference genomes and, optionally, classify the reads as microbial or mammalian.
Example 14. The computer system of Example 13, wherein the instructions further configure the computer system to identify at least one unmapped sequence read of the metagenomic sequence data and, optionally, classify the at least one unmapped read.
Example 15. The computer system of Example 13, wherein mammalian oral microbiome samples having fewer than 10,000 classified microbial reads or more than 500,000 classified microbial reads are excluded from the comparison of the oral microbial profile for the mammalian animal against a database of defined microbial profiles.
Example 16. The computer system of Example 12, wherein the instructions further configure the computer system to calculate an abundance of the one or more microbial species present in the oral sample.
Example 17. The computer system of Example 16, wherein the abundance of the specific one or more microbial species present in the oral sample correlates to whether the specific one or more microbial species is a predictive microbial species for the specific oral disease.
Example 18. The computer system of Example 16, wherein the instructions further configure the computer system to perform a pairwise log ratio comparison of the microbial abundance of the mammalian animal's oral sample against the information in the database.
Example 19. The system of Example 18, wherein the specific one or more microbial species is a predictive microbial species when 50% or more of the maximum possible pairwise log ratio comparisons involving this microbe are significantly different when compared between a disease and a control cohort.
Example 20. A method for predicting the development of an oral disease in a mammalian animal, the method comprising:
   obtaining an oral sample from a mammalian animal, the oral sample containing one or more microbial species;
   isolating, from the oral sample, microbial nucleic acid of the one or more microbial species;
   obtaining microbial nucleic acid sequence data corresponding to the microbial nucleic acid;
   analyzing the microbial nucleic acid sequence data to identify one or more microbial species present in the oral sample and, optionally, quantifying the one or more microbial species;
   generating an oral microbial profile for the mammalian animal based on the identified and, optionally, quantified one or more microbial species, the oral microbial profile comprising the one or more microbial species and, optionally, a quantity or relative abundance of the one or more microbial species in the oral sample;
   comparing the oral microbial profile to information in a database that identifies weighted correlations between:
   (i) occurrence and/or prevalence of one or more oral diseases in animals in a classification of the mammalian animal; and
   (ii) presence and/or abundance of various microbial species in the oral microbiome of animals in the classification of the mammalian animal, wherein the various microbial species comprise the one or more microbial species in the oral sample;
   generating a risk score indicating a likelihood of the mammalian animal developing the one or more oral diseases based on one or more matches between the oral microbial profile and the information in the database; and
   indicating the mammalian animal as developing the one or more oral diseases when the risk score meets or exceeds a predetermined threshold.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Figures, like reference numerals may be utilized to designate corresponding or similar parts in the various Figures, and the various elements depicted are not necessarily drawn to scale, wherein:

FIG. 1A-1B illustrates a dental health test workflow and oral microbiome reference database construction.

FIG. 2A-2C illustrates a distribution of the average log ratio difference scores between pairwise microbial interactions associated with (A) periodontal disease (PD) and healthy cohorts, (B) tooth resorption (TR) and healthy cohorts, and (C) bad breath (BB) and typical breath (TB) cohorts.

FIGS. 3A-3D illustrate sensitivity and specificity of the feline dental health test based on a 2-component Gaussian mixture model.

FIG. 4 illustrates overlap of oral microbiome predictive microbes characteristic of feline periodontal disease, tooth resorption and halitosis.

FIGS. 5A-5B illustrate sampling location effect and reproducibility of the feline dental health test results.

FIG. 6 illustrates microbial species richness as a function of number of sequencing reads, comparing data from two different types of metagenomic whole genome sequencing (WGS) library preparations-a ligation-based approach versus a tagmentation-based approach (such as the Illumina Nextera DNA Flex Library Preparation Kit).

FIG. 7 illustrates an oral microbiome-based periodontal disease risk assessment in clinically recruited cohorts of cats suffering from gingivitis (no alveolar bone loss), periodontal disease and alveolar bone loss, and citizen science recruited healthy controls.

FIG. 8 illustrates an oral microbiome-based bad breath (halitosis) risk assessment in clinically recruited cohorts of cats suffering from gingivitis (no alveolar bone loss), periodontal disease and alveolar bone loss, and citizen science recruited healthy controls.

FIG. 9A illustrates an oral microbiome-based tooth resorption risk assessment in clinically recruited cohorts of cats suffering from tooth resorption and citizen science recruited healthy controls. FIG. 9B illustrates an oral microbiome-based tooth resorption risk assessment in clinically recruited cohorts of cats suffering from tooth resorption, incorporating tooth resorption staging information, and citizen science recruited healthy controls.

FIG. 10A illustrates a distribution of the average log ratio difference scores between pairwise microbial interactions associated with gingivostomatitis and healthy cohorts. FIG. 10B illustrates the sensitivity and specificity of the feline gingivostomatitis test based on a 2-component Gaussian mixture model. Distribution of the probability of cats from the gingivostomatitis and healthy cohorts being classified as having gingivostomatitis or being healthy according to a 2-component Gaussian mixture model. Sensitivity and specificity of the feline gingivostomatitis test based on the ability to detect oral microbiome signatures characteristic of this disease are also shown.

### DETAILED DESCRIPTION

Variations in the microbial composition of the mouth (the oral microbiome) may have associations with certain dental and systemic diseases. This research area is still young and studies on human subjects demonstrating these associations in a comprehensive manner have only been published in the last decade or less. Studies on this topic in companion animals, such as cats and dogs, have been limited. Nutritional and environmental factors, as well as present disease states, may play an important role in the dynamic microbial composition of a mammal's mouth (their oral microbiome). With the mouth being the first line of defense from a constant exposure to foreign microbes, the oral microbiome has evolved to be competitive and territorial. It is comprised of microbes that excel at defending their territory and are typically able to avoid being replaced by foreign invaders, including pathogens. However, dysbiosis inducing events such as poor diet or poor dental hygiene, can lead to pathogenic microbes colonizing disproportionately large parts of the oral cavity (and, thus, altering the oral microbiome), which can be associated with pathology. Understanding the composition of the oral microbiome can provide information about the health of oral tissues and point to potential dental and gum diseases. This information may also be used to manage the health and wellbeing of a pet.

Dental diseases may be associated with complex interactions involving a multitude of microbes, as opposed to a single microbe. The field of oral microbiome research in companion animals has received little focus and it is still in its infancy. Existing studies base their conclusions on small sample sizes and outdated culture-based techniques for querying the microbiome. It is estimated that only around 2% of all existing bacteria can be cultured in the laboratory, meaning that in studies relying on this method for microbial classification, many important microbial organisms will likely be missed, while false emphasis might be placed on particular species, simply because they could be cultured and measured. This problem is compounded by the fact that lab culturing provides a very bacteria-centric view of the microbiome, often ignoring other microorganisms such as fungi, protozoa, archaea and viruses.

Interrogating the oral microbiome of a mammalian animal can be accomplished using an oral (saliva) sample. Saliva sampling kits have gained popularity in recent years as tests for ancestry and microbial infection have become more prevalent. Available direct-to-consumer microbiome tests typically rely on a technique called ‘16S rRNA gene sequencing,’ which utilizes Next Generation Sequencing (NGS). While this technique provides substantially more information than early bacterial culturing efforts, it can only be used for identifying bacterial species (and some archaea) present in the microbiome. In most cases, these tests do not provide sufficient resolution to reliably, and consistently, identify bacteria beyond the genus level of taxonomic classification. Therefore, in most cases, the test results do not provide the exact species or strain of bacteria comprising the microbiome, making data-driven conclusions vague and relying on approximation. Moreover, it is well-known that the microbiomes of different sites of the body can be composed of viruses, protozoa, and fungal species, in addition to bacteria and archaea. This means that the 16S rRNA gene sequencing approach zooms in on just one part of the microbiome, ignoring the rest.

Before describing various embodiments of the present disclosure in detail, it is to be understood that this disclosure is not limited only to the specific parameters, verbiage, and description of the particularly exemplified systems, methods, and/or products that may vary from one embodiment to the next. Thus, while certain embodiments of the present disclosure will be described in detail, with reference to specific features (e.g., configurations, parameters, properties, steps, components, ingredients, members, elements, parts, and/or portions, etc.), the descriptions are illustrative and are not to be construed as limiting the scope of the present disclosure and/or the claimed invention. In addition, the terminology used herein is for the purpose of describing the embodiments and is not necessarily intended to limit the scope of the present disclosure and/or the claimed invention.

Presently disclosed are computer systems, systems and methods for the identification, screening, indication and/or treatment of oral disease states in cats, dogs and/or other mammalian companion animals. Oral disease states are understood to encompass dental disease states, while not being limited to dental disease states. Embodiments of the disclosed subject matter describe a method for interrogating the oral microbiome of a mammalian companion animal for the purpose of detecting microbe compositional abundance trends associated with dental disease in cats, dogs and other mammals. Detecting, identifying and/or quantifying microbe compositional abundance trends enables a practitioner to screen for and/or indicate whether a cat, dog and/or other mammalian animal has a particular oral disease state. Detecting and identifying oral and/or dental disease states enables the practitioner or pet owner to treat and prevent the future recurrence of the dental disease state. Treating and/or preventing oral and/or dental disease states enables treatment and prevention of wider, systemic conditions, beneficially resulting in a healthier and more comfortable life for the pet.

The degree to which the disclosed systems and methods enable detection, identification and indication of disease states such as periodontal disease, is further enabling for the detection, identification and indication of other disease states such as tooth resorption, feline gingivostomatitis and halitosis, among others. Similarly, the degree to which the disclosed systems and methods enable detection, identification and indication of disease states in felines, is further enabling for the detection, identification and indication of disease states in other mammalian animals, such as dogs (and other canines), horses (and other equines), sheep (and other ovines), cows (and other bovines and/or ruminates), pigs (and other porcine animals), guinea pigs, hamsters, etc.

Disclosed methods may compare, for example, a cat's oral microbiome to the oral microbiomes of cats reported by their owners and/or a veterinary professional to have been diagnosed with tooth resorption, periodontal disease, feline gingivostomatitis, or to have bad breath characterized by a ‘death and decay’ odor. The comparison is carried out using a reference database containing defined microbial profiles, associating one or more microbial species and their respective compositional abundance with one or more oral dental conditions.

Disclosed systems and methods can comprise a painless oral swab sample collection. Accordingly, the oral microbiome can be surveyed via buccal, supragingival or subgingival sampling. Such sampling does not require anesthetizing the animal and can be performed by the pet owner at their home or by the veterinarian at the clinic. The disclosed systems and methods can serve as an early indicator of dental disease-associated processes not yet visible to the naked eye or not easily recognizable by the average veterinarian general practitioner who does not have extensive dentistry training. Routine use may enable identification of early-stage dental diseases, driving more pets to the veterinary office early on and reducing the number of emergency dental vet visits in the long run. Earlier identification of oral disease states beneficially saves costs in emergency visits and further saves the lives of mammalian companion animals.

It is already known that the colonization dynamics and influence of microorganisms (and their relative abundance) inhabiting organs and systems, such as the gastrointestinal tract (from mouth to anus), show many similarities between dogs (canines), cats (felines) and humans. Periodontal disease, which is a disease associated with microbial imbalance in the oral cavity, is prevalent among cats, dogs and humans, with some notable commonalities. The porphyromonas and tannerella genera of bacteria, for example, plays a critical role in periodontal disease pathogenesis in cats, dogs and humans. This demonstrated overlap between cats, dogs and humans in both a disease state and microbial culprits suggests there is overlap in additional disease states/microbial culprits in cats, dogs and humans.

The disclosed systems and methods use an oral swab collection device that has previously been successfully used for extracting genomic material from an oral swab sample from either cats or dogs. Additionally, per the manufacturer of the oral swab collection devices, the same swab collection device used is ideal for use with livestock (bovine, ovine, caprine), companion animals (canine, feline, equine) and other species by researchers, breeders, laboratories and consumers. The oral swab collection devices support use across different mammalian species. It has been established that extraction of both host and microbial DNA from such sample collection devices is possible (see the Examples below). It logically follows that the capability for this extraction on feline samples would extend to canine and other mammalian samples.

The disclosed systems and methods demonstrate analysis of microbial species identity and abundance in feline oral microbiome samples for the purpose of screening for dental diseases in cats. Given that dogs and other mammals have oral cavities and oral microbiomes and are, in many cases, predisposed to the same dental disease pathologies (e.g., periodontal disease), our method should be readily applicable to dogs and other mammals. This is because the model for each species is based on a comparison between a disease and a healthy animal cohort in order to derive the precise trends in microbial identities and abundances in each state for each species.

### Defined Microbial Profiles Contained in the Reference Database

With the mouth being the first line of defense from constant exposure to foreign microbes, the oral microbiome has evolved to be competitive and territorial. It is comprised of microbes that excel at defending their territory and are typically able to resist being replaced by foreign invaders, including pathogens. These microbes are generally present when a mammalian animal (e.g., a cat or dog) is healthy and would represent a healthy microbial profile of the oral microbiome. When the mammalian animal is suffering from a dental condition, the composition of the oral microbiome may be altered by the presence of foreign or pathogenic microbial species and/or altered abundance ratios between different microbes. Such an alteration in the composition of the oral microbiome might be represented by a pathogenic profile. In some cases, the presence of particular foreign and/or pathogenic microbial species, and their abundance relative to other microbes in the oral cavity, is correlated with the mammalian animal suffering from a particular dental condition.

Identification of the particular (one or more) microbial species (and their respective relative abundances) correlated with particular oral disease states enables pre-diagnostic screening for the oral disease state in a mammalian animal exhibiting the presence of the identified (one or more) microbial species. In other words, identification and/or indication of the oral disease state may be correlated to the mammalian animal exhibiting a particular pathogenic profile.

The gold standard for the comprehensive study of the microbiome is shotgun metagenomic sequencing, which allows capturing complete or near-complete genomes of organisms across all domains of life, not just bacteria and archaea. The disclosed methods also enable microbial identification and classification down to the species or, in some instances, the strain level, unlike 16S gene sequencing.

In veterinary practice, dental disease is sometimes thought of as a syndrome where halitosis, tooth resorption and periodontal disease are rarely seen separately from each other, even though they can have different underlying pathologies and/or microbial culprits. As discussed more fully below, this view is, to some extent, reflected in obtained data where some overlap in microbial species between conditions is observed. The largest overlap observed is between halitosis and periodontal disease, which is consistent with observations from the clinic where halitosis is often a harbinger of periodontal disease. However, also identified were a plethora of microbes whose compositional abundance in the oral microbiome is predictive specifically of halitosis, tooth resorption, feline gingivostomatitis or periodontal disease. This suggests that there are microbial profiles associated with specific dental pathologies, in addition to the existence of a core set of microbes associated with dental disease in general.

Using shotgun metagenomic oral microbiome sequencing of 38,000 domestic cats and compositional data analysis techniques, a comprehensive survey of the feline oral microbiome was executed, identifying 8,344 microbial species present in the feline oral microbiome. Whether a domestic cat included in the shotgun metagenomic sequencing suffered from a particular dental condition was identified in two ways. Cats were either reported by their owners to have been formally diagnosed by a veterinarian as suffering from a particular dental condition (e.g., periodontal disease, tooth resorption, gingivostomatitis, etc.) or were informally diagnosed by their owner (as is the case for halitosis).

The reference database is a weighted correlation database and contains at least the identified 8,344 microbial species present in the feline oral microbiome. On average, 606 microbial species per cat were identified, 97% of which were classified as bacteria and archaea, 0.27% as DNA viruses (RNA viruses cannot be detected with shotgun metagenomic sequencing), 0.02% as phages and <2% as fungi. The various microbial species identified as being involved in and contributing to a specific dental disease are compiled into a “defined microbial profile.” The defined microbial profile is a list or collection of identified one or more microbial species and their respective relative abundances known to contribute to and/or be involved in a specific dental disease condition.

For example, a defined microbial profile may include a set of 27 microbes that are predictive for three dental conditions (halitosis, tooth resorption and periodontal disease), as well as microbes specifically predictive for one of the four dental conditions (halitosis, feline gingivostomatitis, tooth resorption and periodontal disease). “Predictive microbes” are discussed more fully below. The defined microbial profile may rank and/or weigh each included microbial species by how frequently and in what proportions a certain microbe is observed in animals suffering from the specific dental disease condition, as deduced by consulting a reference database. How much any one microbial species contributes to a specific dental disease condition is correlated to how often a microbial species shows up (or is present) in the oral microbiome while an animal is suffering from a specific dental disease condition, as well as how consistently such microbial species demonstrates significantly different relative abundance from other oral microbes when compared to healthy control samples.

The defined microbial profiles contained in the reference database also include defined microbial profiles of healthy mammalian animals that are not suffering from a dental condition. For example, the defined microbial profile of healthy cats lists and identifies the microbial species present in the oral microbiome, as well as their relative abundances, when no dental condition is present. A healthy defined microbial profile may establish a baseline or control for the microbial species present and their relative abundance. Any deviations from this profile may enable a practitioner to predict and/or indicate, for example, a cat's likelihood of suffering from a dental condition. Similarly, deviations from the healthy defined microbial profile may enable a practitioner in diagnosing a cat as suffering from a dental condition prior to the onset of symptoms for that dental condition.

The defined microbial profile for each dental disease state is compared to the defined microbial profile for a healthy mammal to determine any differences between the dental disease states and a healthy state. In some embodiments, the comparisons are pairwise log ratio comparisons. For example, there may be some overlap in the oral microbiome of a healthy cat and a cat suffering from periodontal disease. A comparison of the healthy defined microbial profile to the periodontal disease defined microbial profile would identify common microbial species seen in similar abundances between the two. Any microbial species not common between the two microbial profiles, or any microbial species seen in significantly different proportions between the two profiles, would confirm the involvement of that microbial species in the development of periodontal disease. Identification of such a microbial species in a cat's oral microbiome would be indicative of the cat having periodontal disease.

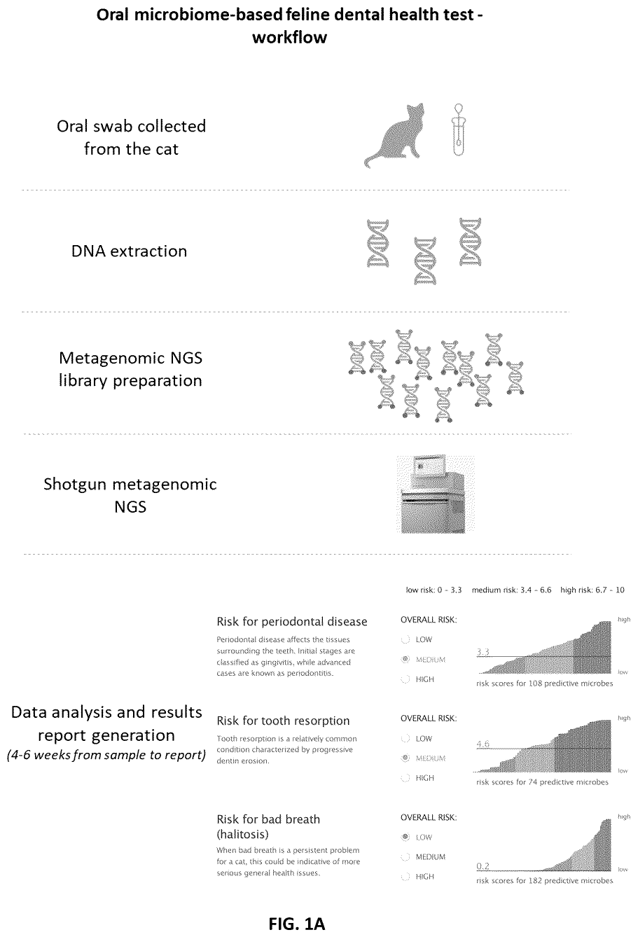

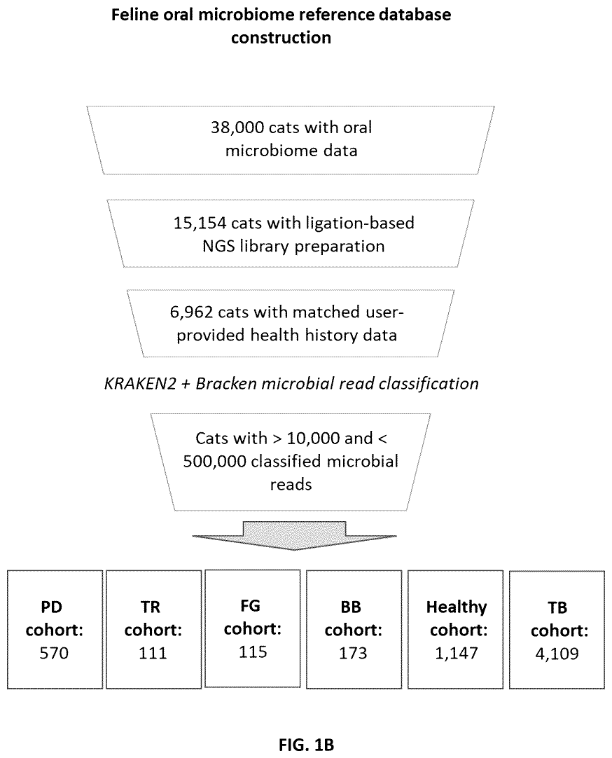

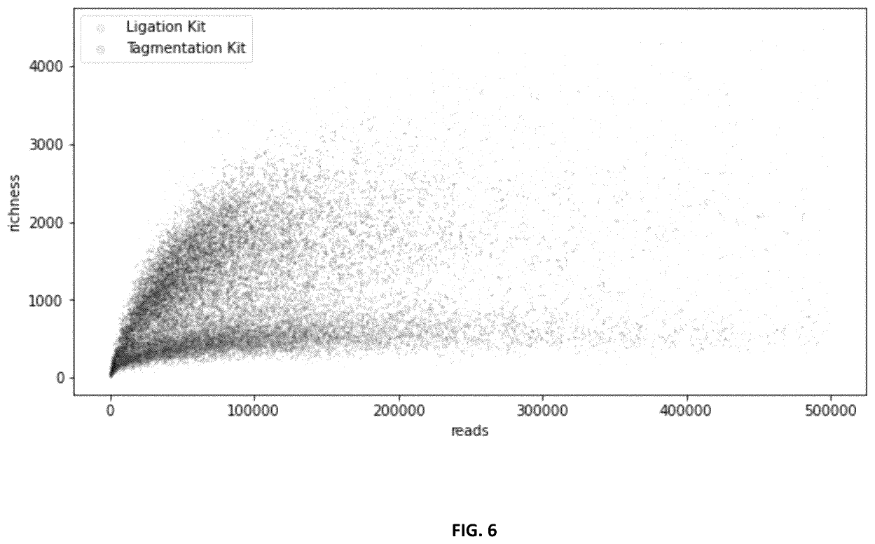

FIGS. 1A-1B illustrate a dental health test workflow and construction of the oral microbiome reference database using feline subjects. In FIG. 1A, the feline dental health test workflow includes collecting an oral swab from the cat in a DNA preservation solution, extracting and preparing the DNA for shotgun metagenomic next generation sequencing (NGS), sequencing, data analysis and the generation of a report presenting risk assessment for different dental diseases based on the state of the oral microbiome, accompanied by treatment recommendations tailored to the results. In FIG. 1B, the feline oral microbiome reference database was constructed through applying sequential filters on the initial database of 38,000 cats. First, all data from tagmentation-based NGS library preparation samples was removed. This was done due to an observed effect of the library preparation method on microbial species richness (FIG. 6). The ligation-based method was preferred because the number of sequencing reads per sample had minimal impact on the number of microbial species detected. In addition, Tn5 transposase assisted tagmentation is known to introduce GC sequencing bias, particularly in metagenomic communities. However, tagmentation-based NGS library preparation may be included in some embodiments.

Next, samples lacking an accompanying phenotype/health history record for the cat were excluded. After classification of the microbial reads in each sample using KRAKEN2 and Bracken, samples with fewer than 10,000 and more than 500,000 classified microbial reads were removed. The remaining cats/samples were placed into cohorts. This resulted in a periodontal disease (PD) cohort of 570 cats, tooth resorption (TR) cohort of 111 cats, feline gingivostomatitis (FG) cohort of 115 cats, bad breath (BB) cohort of 173 cats, healthy cohort of 1,147 cats and typical breath (TB) cohort of 4,109 cats.

### Identifying Predictive Microbes

As a first step towards identifying microbes significantly correlated with each dental condition, Pairwise Log-Ratio (PLR) transformation was performed on the Bracken output species level read counts. Next, the significant PLR comparisons (p-value <0.01) were identified between the control and a condition by performing a z-test. The healthy cohort was compared to the PD, TR and FG cohorts; the typical breath (TB) cohort was compared to the BB cohort.

The frequency of each microbial species in all significant PLRs was assessed. Only microbial species where 50% or more of their maximum possible comparisons with other species were significant were kept. This measure was used as a proxy for the importance of different microbial species in the four dental conditions of interest. These microbial species are “predictive microbial species” for each dental condition.

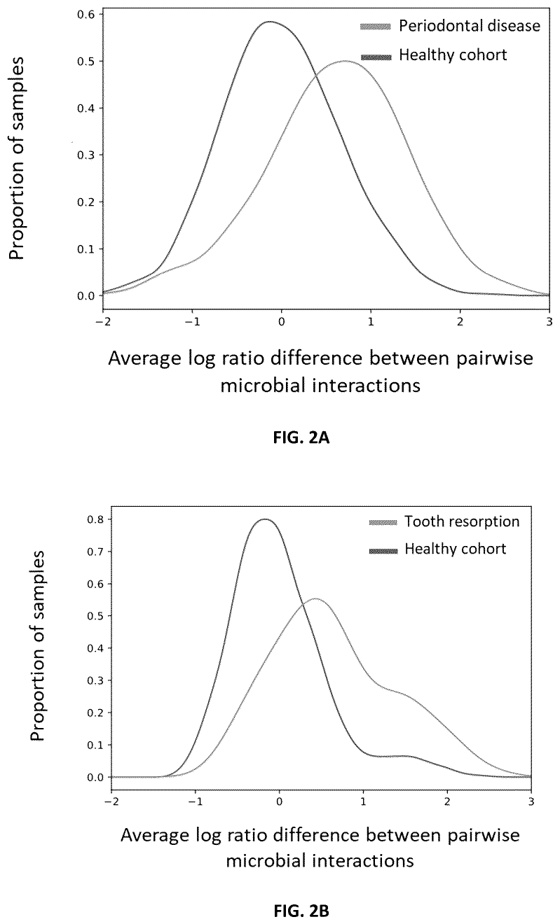

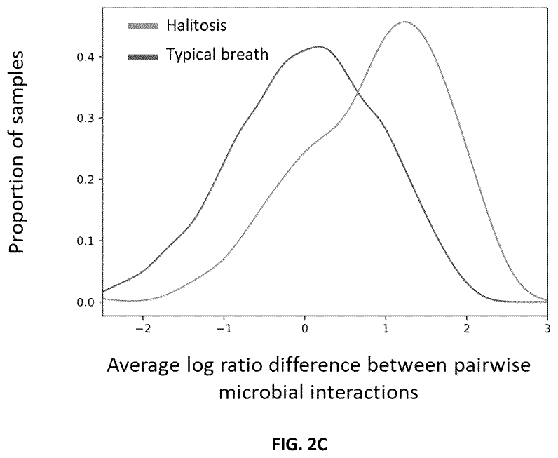

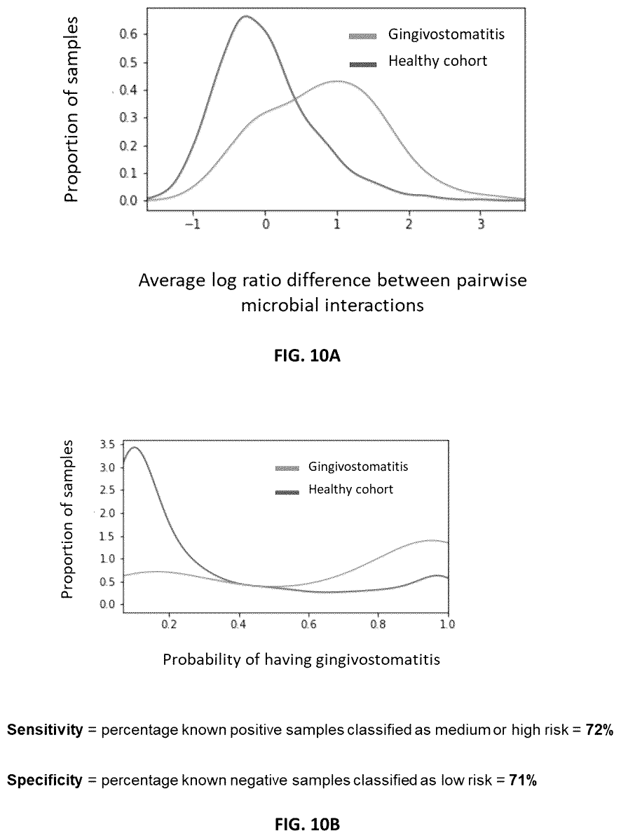

In order to identify population-wide microbial compositional abundance patterns characteristic of periodontal disease, tooth resorption, feline gingivostomatitis, or halitosis, for each of the conditions, each sample was scored by comparing the predictive pairwise log-ratios (pPLRs) of the sample to the mean pPLRs of controls, taking into account the direction and magnitude of the difference. FIGS. 2A-2C illustrate a distribution of the average log ratio difference scores between pairwise microbial interactions associated with periodontal disease and healthy cohorts, tooth resorption (TR) and healthy cohorts, and bad breath (BB) and typical breath (TB) cohorts. FIG. 10A illustrates the distribution of the average log ratio difference scores between pairwise microbial interactions associated with feline gingivostomatitis (FG) and healthy cohorts.

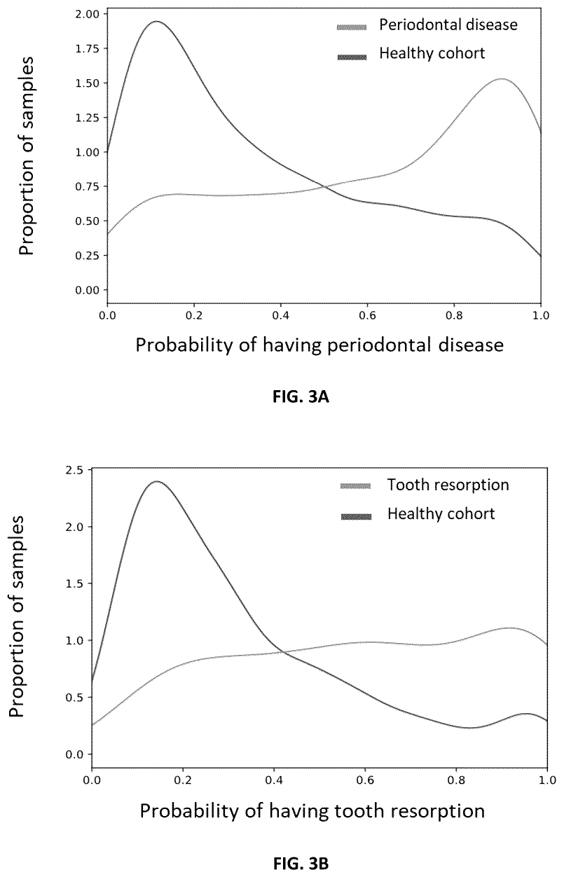

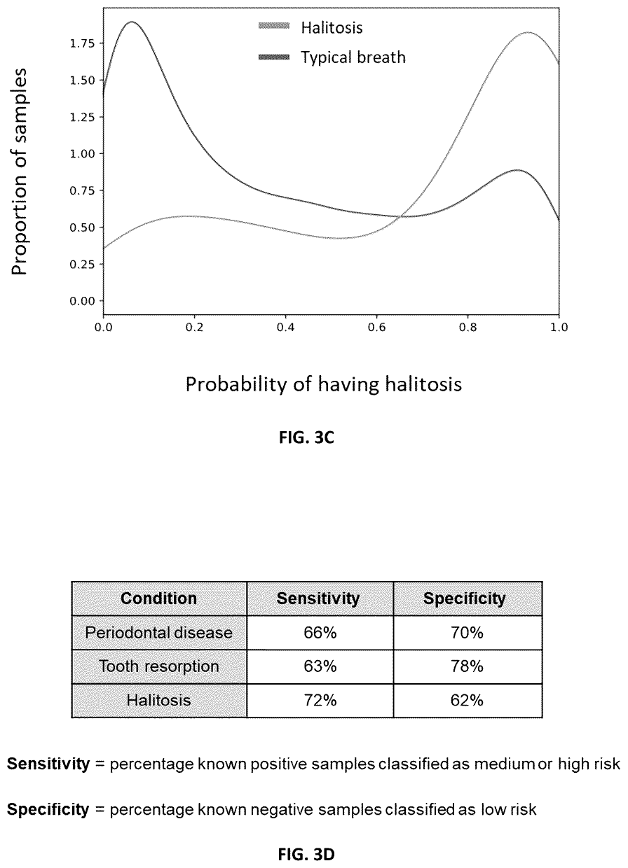

Next, we fitted four (4) Gaussian mixture models (one for each dental condition) with two (2) components each-healthy cohort (or TB cohort) and dental condition-onto the distribution of the average log ratio difference score between pairwise microbial interactions. This modeling approach generates a 0 to 1 score for each sample, which represents the probability that the sample belongs to the control cohort or to the respective dental condition cohort. FIGS. 3A-3C plot the probability that samples belonging to three of the dental disease cohorts (periodontal disease, tooth resorption and halitosis) and the control samples would be classified as belonging to their respective cohorts based on each sample's compositional abundance of predictive microbes. FIG. 10B plots the probability that feline gingivostomatitis and control samples would be classified as belonging to the feline gingivostomatitis category or to the control category based on each sample's compositional abundance of predictive microbes. A bimodal probability distribution consistent with sample identity was observed between the dental condition and control in all cases. The clearest bimodal pattern was for periodontal disease and halitosis, and a weaker bimodal pattern for tooth resorption and feline gingivostomatitis was observed. In all four instances, there was a minority of disease samples forming a small peak closer to 0 and a small set of control samples forming a slight peak closer to 1.

The defined microbial profile for each dental disease state (periodontal disease, tooth resorption, feline gingivostomatitis and halitosis) is compared to the defined microbial profile for a healthy mammal to determine and quantify differences and commonalities in microbial species and their abundance between the dental disease states and a healthy state. The defined microbial profiles for each dental disease state are also compared to each other to identify overlapping microbial species common to each dental disease state.

The defined microbial profiles for each dental disease state and a healthy control state undergo a pairwise log ratio (PLR) transformation. The PLR transformation corrects for potential sequencing coverage differences between samples by scaling microbial abundances relative to each microbe instead of a constant scaling factor. Next, a z-test between PLRs from each disease state versus the control state is performed. A p-value of approximately <0.01 serves as a threshold value for significant PLR comparisons. For each microbial species identified in a defined microbial profile for a dental disease state, the number of significant PLR comparisons (as defined by the p-value) that microbial species shows up in is counted. If the number of significant PLR comparisons is at least 50% of all possible PLR comparisons for that microbe, the microbial species is deemed a “predictive microbe.” This process may be repeated for each dental disease state of interest. In other words, through z-test identification of significant PLR comparisons, predictive microbes can be identified for periodontal disease, halitosis, feline gingivostomatitis and/or tooth resorption. Table 2 provides examples of identified predictive microbes for periodontal disease, halitosis and tooth resorption.

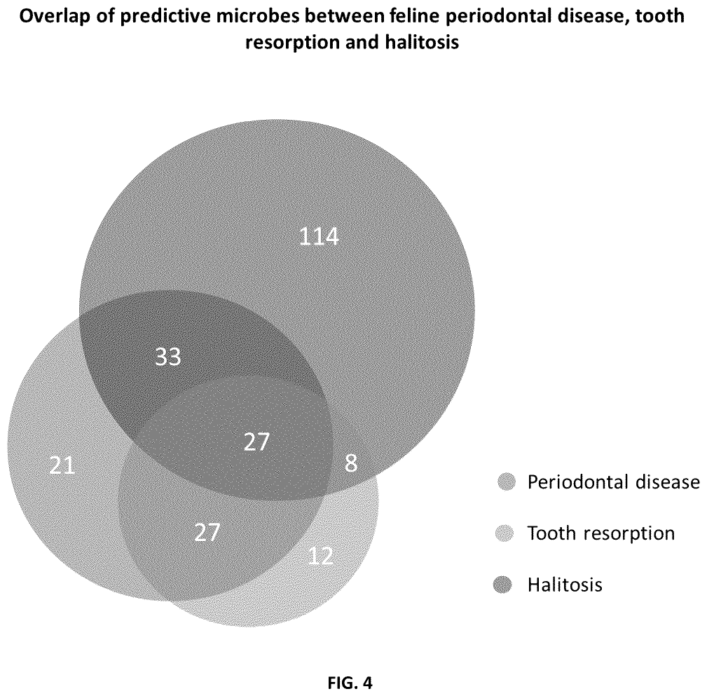

As outlined in Table 2, 108 predictive microbes for periodontal disease, 74 for tooth resorption and 182 for halitosis were identified. The predictive microbes for each dental disease were identified based on PLR microbial abundance comparisons between healthy/control defined microbial profiles and the defined microbial profiles of cats suffering from one of three dental conditions (See FIG. 4). 27 microbes were identified as predictive for three dental conditions (periodontal disease, tooth resorption and halitosis), though each condition has its own specific set of predictive microbes, differentiating it from other conditions. Plotting the average log ratio difference between significant pairwise microbial interactions in a dental condition versus control samples allowed separation of sample populations based on their dental disease status. (See FIGS. 2A-2C). However, some overlap between the populations was observed, meaning that for a certain set of samples, their compositional abundance of predictive microbes could be interpreted as either consistent with the control population or the respective dental disease population.

It is important to note that the use of the word ‘predictive’ is not meant to be interpreted as ‘causative’, it simply reflects the fact that a microbe has a significantly different compositional abundance in a particular dental condition compared to control. This could either mean that the microbe has an active role in the disease's pathology or that the changes of its compositional abundance are a byproduct of pathology. In either scenario, presence of the microbe in a specific abundance relative to other microbes directly correlates with an oral and/or dental disease state.

A significantly increased compositional abundance of P. gingivalis, T. forsythia, B. zoogleoformans, D. orale, D. fairfieldensis and T. denticola (among other microbes) was observed in the microbiomes of cats suffering from periodontal disease. Furthermore, significantly decreased compositional abundance of the genera Moraxella and Capnocytophaga, as well as the bacterial species P. multocida, was also observed (Table 1). These observations are all consistent with previous findings from studies focused on the oral microbiome of cats, humans and dogs suffering from periodontal disease.

### Sequencing and Extraction Protocols

At least one oral swab of a mammalian animal may be taken to provide a sample for testing. The oral swabs may target the gum lines of the animal (top and bottom) and/or target the entire mouth of the animal. Microbial DNA may be extracted from the oral swab samples in order to identify which microbial species, and in what relative abundance, are present in the mammalian animal's oral microbiome.

Metagenomic DNA may be extracted from the oral samples via heat treatment for approximately one hour on a shaker, with or without bead-beating or the addition of detergents and protein degradation reagents such as proteinase K. In some embodiments, the oral samples are heat treated at approximately 45° C. to 75° C., such as 50° C., 55° C., 60° C., 65° C., 70° C. or within a range defined by any two of the foregoing values.

After heat treatment of the oral sample, metagenomic DNA may be extracted by SPRI magnetic beads-based DNA extraction (MCLAB, MBC-200) using 80% ethanol for purification. The DNA may be quantified using a GloMax Plate Reader (Promega). Following metagenomic DNA extraction and quantification, the oral samples may be prepared for NGS using the LOTUS DNA library prep kit (IDT), the Next Ultra II FS DNA library prep kit (NEB), or another ligation or tagmentation based DNA library prep kit, following the manufacturer's instructions. The oral samples may be dual-barcoded with iTRU indices. The prepared sequencing libraries may be quantified using a GloMax Plate Reader (Promega) and equal-mass pooled into 96-sample pools. The pools may then be visualized (to assess fragment size distribution) and quantified using a 2100 Bioanalyzer instrument (Agilent). Following standard QC steps, the 96-sample pools may be loaded onto an Illumina HiSeq X or NovaSeq 6000 Next Generation Sequencing machine. The raw sequencing data may be demultiplexed and trimmed to remove low-quality

data using, for example, the program Trimmomatic 0.32. The data may then be mapped to the latest version of, for example, the feline genome Felis_catus_9.0, or the reference genome of the mammalian species of interest. For every oral sample, there may be approximately 5-7% sequencing reads that do not map to the mammalian genome of interest. The unmapped reads may be classified using the KRAKEN2 metagenomic sequence classifier (or a suitable alternative) to identify the microbial organisms present in each sample. Bracken, a statistical method for calculating species abundance in DNA sequencing data from a metagenomic sample, was used on the sequenced data in conjunction with the KRAKEN2 analysis. Bracken may output species level read counts. Based on the outcome of the KRAKEN2 metagenomic sequence classifier and the Bracken calculations, an oral microbial profile for the mammalian animal may be generated. The oral microbial profile generated may include data regarding the identity of the microbial species present as well as their relative abundance.

A confidence score of approximately 0.1 may be used as a cutoff (or threshold value) for the KRAKEN2 classification algorithm. All samples with fewer than 10,000 classified microbial reads or more than 500,000 classified microbial reads may be filtered out. The reads for microbial species with a non-zero mean of fewer than 10 reads may also be filtered out.

### Methods of Indication and Comparison

Indication of whether a cat is suffering from a dental disease relies on a comparison of the cat's current oral microbiome state to the oral microbiomes of cats reported by their pet owners to have been diagnosed by a veterinarian with periodontal disease, gingivostomatitis or tooth resorption or to suffer from bad breath (halitosis). The comparison is based on the compositional abundance of microbes determined by the analysis to be predictive of each of the three dental conditions.

Computational analysis of the compositional abundance of different microbes present in the oral microbiome involves comparison of the sample against a database of samples from mammals of the same species known to suffer from different dental conditions, as well as mammals of the same species who do not suffer from any known dental conditions. In other words, the computational analysis compares the oral microbiome identified from the oral swab sample to the defined microbial profiles contained in the reference database (discussed more fully above).

In some embodiments, a method for indicating oral disease in mammalian animals includes receiving an oral swab sample taken from a mammalian animal; performing heat treatment on the oral sample; and performing magnetic beads-based deoxyribonucleic acid (DNA) extraction on the heat-treated oral sample to extract microbial DNA that is present in the oral swab sample. The method may also include sequencing the microbial DNA to identify which specific one or more microbes are present in the oral sample and in what proportions, wherein identifying the specific one or more microbes and their abundances results in generation of an oral microbial profile for the mammalian animal; and comparing the oral microbial profile for the mammalian animal against a database of defined microbial profiles, wherein the database identifies correlations between (i) profiles that include one or more microbes and (ii) corresponding oral diseases.

Based on a result of comparing the oral microbial profile against the database of defined microbial profiles, the method may further include generating a risk score indicating a likelihood that the mammalian animal has a specific oral disease.

In some embodiments, a method for indicating oral disease in mammalian animals includes receiving an oral swab sample taken from a mammalian animal; performing heat treatment on the oral sample; and performing magnetic beads-based deoxyribonucleic acid (DNA) extraction on the heat-treated oral sample to extract microbial DNA that is present in the oral swab sample. The method may also include sequencing the microbial DNA to identify which specific one or more microbes are present in the oral sample, wherein identifying the specific one or more microbes and their abundance results in generation of an oral microbial profile for the mammalian animal.

The method may further include comparing the oral microbial profile for the mammalian animal against a database of defined microbial profiles, wherein the database identifies correlations between (i) profiles that include one or more microbes and (ii) corresponding oral diseases; based on a result of comparing the oral microbial profile against the database of defined microbial profiles, generating a risk score indicating a likelihood that the mammalian animal has a specific oral disease; and in response to generating the risk score and identifying the specific oral disease, administering a therapeutic treatment designed to treat the specific oral disease.

In some embodiments, the therapeutic treatment may include administering a therapeutic compound, such as a compound designed to inhibit or encourage growth of a specific one or more microbial species present in the oral microbiome of the mammal. In some embodiments, the therapeutic compound includes a pre-biotic, a post-biotic, a pro-biotic, a medicament or a combination thereof. In some embodiments, the therapeutic treatment may include brushing the mammal's teeth with a topical treatment.

In some embodiments, the therapeutic treatment protocol is designed to alter the composition of the oral microbiome of the mammal. In some embodiments, altering the composition of the mammal's oral microbiome treats and/or addresses the specific oral disease. In some embodiments, the therapeutic treatment repairs the mammal's oral microbiome. In some embodiments, repairing the mammal's oral microbiome brings the mammal's oral microbiome more in line with the oral microbiome (or defined oral microbial profile) of a healthy mammal—both in terms of the specific one or more microbial species present and their relative abundance. In some embodiments, the therapeutic treatment protocol is designed to maintain the composition of the oral microbiome of the mammal. In some embodiments, the therapeutic treatment protocol is designed to stimulate a metabolic output of the mammalian animal's oral microbiome. Stimulating a metabolic output of the mammalian animal's oral microbiome may include using known enzymatic pathway analysis tools to provide an additional dimension to the existing microbial composition data to further characterize disease signatures and improve predictive disease models.

### EXAMPLES

The examples below were performed with cats/feline data, though it is to be understood that the methods outlined are expected to be accurate and appropriate with regards to other mammalian data (e.g., canine, or another mammal).

### Confirmatory Study of the Database

To assess over- or under-representation of particular microbial species in periodontal disease, the Bracken output data underwent Centered Log-Ratio (CLR) transformation. This was done to account for potential compositional biases, which are a well-established problem in microbiome data analysis. A z-test was then performed on the CLR transformed data to identify microbial species with statistically significant increased and decreased compositional abundance in periodontal disease compared to control. Table 1 shows microbes with upregulated and downregulated abundance in the periodontal disease cohort compared to control. The results expand on and agree with previous findings from oral microbiome studies of periodontal disease in humans, dogs and cats. These results validate the sample collection, DNA extraction, metagenomic sequencing and compositional abundance-based analysis methodology. These results also enable a similar identification of microbes with upregulated and downregulated abundances in other disease states, such as halitosis, gingivostomatitis and tooth resorption.

To start building computational oral disease classification algorithms, Pairwise Log-Ratio (PLR) transformation was performed on the Bracken output species level read counts. Next, the significant PLR comparisons (a threshold p-value <0.01) were identified between the control and a condition by performing a z-test. The healthy cohort was compared to the PD, TR and FG cohorts; the typical breath (TB) cohort was compared to the BB cohort.

The frequency of each microbial species in all significant PLRs was assessed. Only microbial species where 50% or more of their maximum possible comparisons with other species were significant were kept. This measure was used as a proxy for the importance of different microbial species in the three dental conditions of interest. These microbial species are “predictive microbial species” for each dental condition.

In order to identify population-wide microbial compositional abundance patterns characteristic of periodontal disease, tooth resorption, feline gingivostomatitis, or halitosis, for each of the conditions, each sample was scored by comparing the predictive pairwise log-ratios (pPLRs) of the sample to the mean pPLRs of controls, taking into account the direction and magnitude of the difference. FIGS. 2A-2C illustrate a distribution of the average log ratio difference scores between pairwise microbial interactions associated with periodontal disease and healthy cohorts, tooth resorption (TR) and healthy cohorts, and bad breath (BB) and typical breath (TB) cohorts. FIG. 10A illustrates the distribution of the average log ratio difference scores between pairwise microbial interactions associated with feline gingivostomatitis (FG) and healthy cohorts.

Next, we fitted four (4) Gaussian mixture models (one for each dental condition) with two (2) components each—healthy cohort (or TB cohort) and dental condition—onto the distribution of the average log ratio difference score between pairwise microbial interactions. This modeling approach generates a 0 to 1 score for each sample, which represents the probability that the sample belongs to the control cohort or to the respective dental condition cohort. FIGS. 3A-3C plot the probability that samples belonging to three of the dental disease cohorts (periodontal disease, tooth resorption and halitosis) and the control samples would be classified as belonging to their respective cohorts based on each sample's compositional abundance of predictive microbes. FIG. 10B plots the probability that feline gingivostomatitis and control samples would be classified as belonging to the feline gingivostomatitis category or to the control category based on each sample's compositional abundance of predictive microbes. A bimodal probability distribution consistent with sample identity was observed between dental condition and control in all cases, with the clearest bimodal pattern for periodontal disease and halitosis and a weaker bimodal pattern for tooth resorption and feline gingivostomatitis. In all four instances, there was a minority of disease samples forming a small peak closer to 0 and a small set of control samples forming a slight peak closer to 1.

This suggests that it is possible that a small proportion of cats in the dental disease cohorts might actually be healthy or in remission (due to old, wrong or incomplete health information provided by the pet owner), while some cats in the control cohorts could be suffering from a dental condition that has not yet been diagnosed or noticed. The sensitivity (ability to detect cats known to suffer from a dental condition) and specificity (ability to detect cats in the control cohort as not suffering from a dental condition) of the risk classification method for each dental condition was tested (see FIG. 3D and FIG. 10 B). The method's sensitivity is highest for halitosis and gingivostomatitis and lowest for tooth resorption, while the specificity is highest for tooth resorption and lowest for halitosis.

This relatively low sensitivity for tooth resorption could be due to the nature of the pathology behind this condition. It tends to originate inside of the tooth and, as it enters more advanced stages, it then reaches the surface of the tooth. It is possible that the microbes associated with tooth resorption can be detected most reliably when the resorptive process has reached the surface of the tooth.

The specificity for periodontal disease and bad breath is lower (70% and 62%, respectively) compared to the specificity for detecting tooth resorption associated changes in the microbiome (78%). This observation could be explained by the possibility that the healthy and TB cohorts include some cats with periodontal disease or bad breath, respectively, that have not yet been diagnosed by a veterinarian or noticed by the pet owner. However, even with these caveats in mind, the specificity and sensitivity of the disclosed methods for all four conditions are comparable to (or better than) previously reported human microbiome-based disease risk assessment algorithms.

Even though a sizable domestic cat cohort (n=6,110) was used to develop the reference database, the health history data for these cats was provided by the pet owner. Despite the fact that pet owners were asked if their cats had been diagnosed by a veterinarian with periodontal disease, gingivostomatitis or tooth resorption, some of the diagnostic precision would have, undoubtedly, suffered, having been relayed by the pet owner. To alleviate this problem and limit instances where a cat reported by their pet owner to be healthy (i.e., not suffering from any known systemic or dental conditions) had actually started developing a yet undiagnosed dental disease, an age limit was set to the control healthy cohort of 1-3 years. This limit was set due to the well-established connection between age and dental disease. Cats below one year of age were intentionally excluded from this group with the purpose of avoiding any potential kitten-specific oral microbiome bias.

The healthy control cohort could potentially be biased towards the oral microbiomes of younger cats and not be representative of older cats with no dental or systemic diseases. The assessment of whether cats in the BB and TB cohorts had halitosis or not was based on the subjective evaluation of the pet owner, which could have potentially added another source of bias.

### Study 1

The present study was of observational nature and did not utilize any invasive procedures. All feline oral swab samples and accompanying health history information used in this study were provided voluntarily by pet owners who agreed in electronic form for their cat's data to be used in an aggregated de-identified format for research purposes. Participants were recruited through an email inviting participation in studies focused on feline dental health.

Twelve (12) cats took part in Study 1. The pet owners of the feline participants received two (2) DNAGenotek's PERFORMAgene (PG-100) oral swab collection devices and were instructed to swab their cat once using each swab. The first swab was used to collect a sample from the whole mouth, while the second one targeted the gum line specifically. Participants were also asked to collect the two samples in one sitting.

The vast majority of feline oral swab samples were collected by pet owners at their respective homes, with a small proportion of sample collections performed by a veterinarian. Pet owners and veterinarians were instructed to collect the samples at least 30 minutes to an hour after the cat had had anything to eat or drink. They were also instructed to keep the oral swab sample collection device in the cat's mouth for at least 5 seconds.

Metagenomic DNA was extracted from feline oral samples via heat treatment (55° C.) for an hour on a shaker, followed by SPRI magnetic beads-based DNA extraction (MCLAB, MBC-200) using 80% ethanol for purification. The DNA was quantified using a GloMax Plate Reader (Promega). Following metagenomic DNA extraction and quantification, each sample was prepared for NGS using the LOTUS DNA library prep kit (IDT) following the manufacturer's instructions. Each sample was dual-barcoded with iTRU indices. The prepared sequencing libraries were quantified using a GloMax Plate Reader (Promega) and equal-mass pooled. The pools were then visualized (to assess fragment size distribution) and quantified using a 2100 Bioanalyzer instrument (Agilent).

Following standard QC steps, the sample pool was loaded onto an Illumina HiSeq X or NovaSeq 6000 Next Generation Sequencing machine. The raw sequencing data was demultiplexed and trimmed to remove low-quality data using the program Trimmomatic 0.32. The data was then mapped to the latest version of the feline genome Felis_catus_9.0. For every sample, there were 5-7% sequencing reads that did not map to the feline genome. The unmapped reads were classified using the KRAKEN2 metagenomic sequence classifier to identify the microbial organisms present in each sample. A confidence score of 0.1 was used as a cutoff for the KRAKEN2 classification algorithm. One sample from a cat undergoing antibiotic treatment at the time of sample collection had fewer than 10,000 classified microbial reads and was therefore excluded from the analysis. Bracken, a statistical method for calculating species abundance in DNA sequencing data from a metagenomic sample, was used on the sequenced data.

Each cat's risk of having tooth resorption, periodontal disease or halitosis was calculated based on the pattern of predictive microbe PLRs observed in their oral microbiome. Briefly, the Bracken output microbial abundance data for each sample was transformed into PLRs and the PLRs associated with predictive microbes were compared to the mean predictive microbes PLRs for the healthy reference cohort. This comparison resulted in a list of deviation scores from the healthy cohort mean for each predictive microbe PLR in the sample of interest. This list of deviations was compared to the list of mean deviations in predictive microbe PLRs for a disease cohort of interest (e.g., tooth resorption, periodontal disease or halitosis) from a healthy control cohort with the purpose of assessing whether the sample shows a similar deviation profile from the healthy cohort as does the reference disease cohort.

Assessing this similarity takes into account the directionality of deviations for each predictive microbe PLR, ‘punishing’ deviations that are in the opposite direction of the respective PLR deviation in the disease cohort compared to the healthy cohort. In other words, if a PLR deviation is in the opposite direction, this is assumed to bring the sample closer to the healthy profile. After summing up all the sample PLR deviation scores, the final deviation score was transformed to fit the probability space in the 2-component Gaussian model for the disease of interest versus healthy. Therefore, each sample had a probability score between 0 and 1 for each dental condition. The following three (3) risk assessment categories based on the probability score generated for each sample were applied: the 0.0-0.33 bracket is classified as ‘low risk’ of having a dental condition; >0.33-0.66 is classified as ‘medium risk’ for having a dental condition; and >0.66-1.0 is classified as ‘high risk’ for having a dental condition.

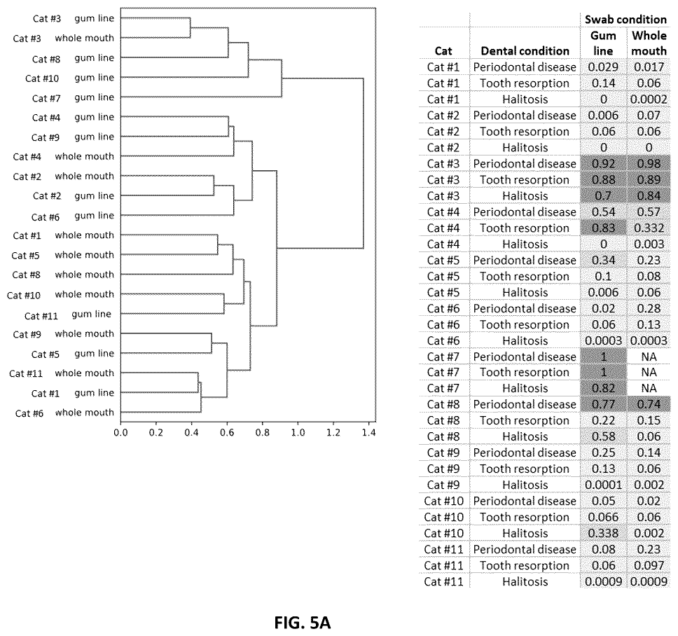

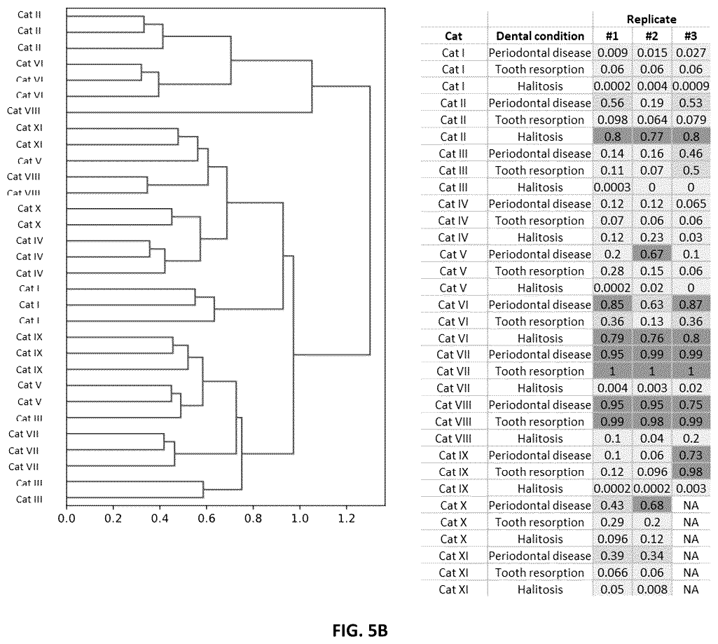

FIG. 5A illustrates results from Study 1 comparing the oral microbiome profiles of 11 cats based on sample collection methods targeting the whole mouth area or the gum line specifically. The dendrogram shows sample clustering based on Spearman's rank correlation of the oral microbiome profiles. The table shows each participating cat's risk assessment for periodontal disease, tooth resorption and halitosis based on the swabbing condition. Green color indicates low risk, light orange-medium risk and dark orange-high risk. Cat #7's ‘whole mouth’ sample was excluded from the analysis because the number of classified microbial reads was <10,000.

### Study 2

The present study was of observational nature and did not utilize any invasive procedures. All feline oral swab samples and accompanying health history information used in this study were provided voluntarily by pet owners who agreed in electronic form for their cat's data to be used in an aggregated de-identified format for research purposes. Participants were recruited through an email inviting participation in studies focused on feline dental health.

Eleven (11) cats took part in this study. Participants received three (3) DNAGenotek's PERFORMAgene (PG-100) oral swab collection devices and were instructed to perform three oral swab collections targeting the whole oral cavity, with all three swab collections performed in one sitting.

The vast majority of feline oral swab samples were collected by pet owners at their respective homes, with a small proportion of sample collections performed by a veterinarian. Pet owners and veterinarians were instructed to collect the samples at least 30 minutes to an hour after the cat had had anything to eat or drink. They were also instructed to keep the oral swab sample collection device in the cat's mouth for at least 5 seconds.

The feline oral swab samples underwent the same extraction, sequencing and analysis protocols outline above for Study 1.

FIG. 5B illustrates the results from Study 2 comparing the oral microbiome profiles of 11 cats based on three separate sample collections targeting the whole mouth, not just focusing on the gum line. The dendrogram shows sample clustering based on Spearman's rank correlation of the oral microbiome profiles. The table shows each participating cat's risk assessment for periodontal disease, tooth resorption and halitosis based on each replicate. Some owners only provided two (2) samples rather than the requested three (3); and some samples were excluded because the number of classified microbial reads was <10,000.

### Study 3

Following obtainment of written consent from pet owners, thirty-six (36) feline oral swab samples from felines (cats) suffering from various degrees of periodontal disease and tooth resorption were collected by a licensed veterinary technician at a feline-only animal hospital using DNAGenotek PERFORMAGENE P-100 collection devices, with the sample collection method targeting the gum line (gingiva). Each cat participating in this trial had accompanying veterinary records and dental radiographs performed within a week of sample collection.

A boarded veterinary dentist blindly assessed the dental radiographs for each sample to confirm diagnosis. The cats were categorized into three groups-cats with mild/moderate gingivitis and no radiographic evidence of alveolar bone loss (n=10), cats with periodontal disease and radiographic evidence of bone loss (n=11) and cats with radiographic evidence of tooth resorption, stages 2-4, affecting one or more teeth (n=15). DNA was extracted from these samples, after which shotgun metagenomic sequencing was performed and the data was analyzed using the computational dental disease risk assessment methods and/or computer systems described previously. The algorithm-produced dental disease risk assessments for these clinically recruited cohorts of cats were compared against risk assessments for a randomly selected cohort of cats whose owners had reported them to not have been diagnosed by a veterinarian with any dental disease and to have fresh or typical cat breath (healthy citizen science-based cohort). Basic demographic statistics of all cohorts are presented in Table 3.

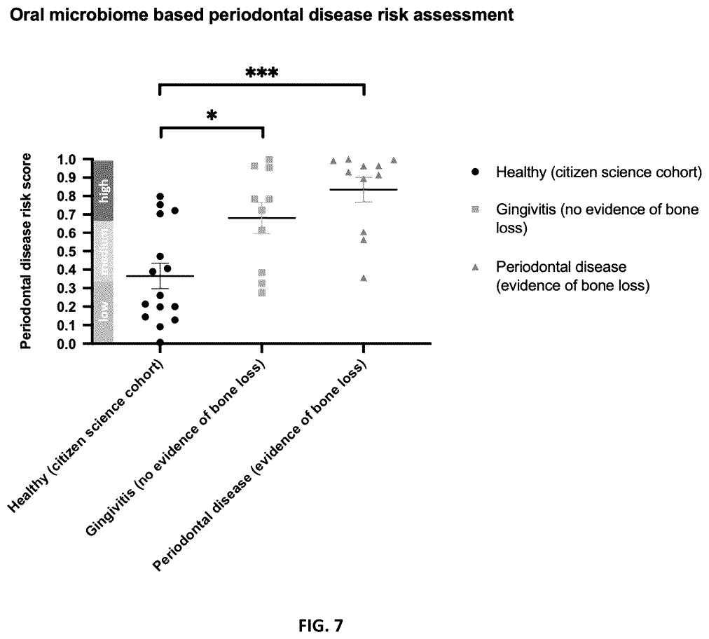

The generated periodontal disease risk assessment was significantly higher for the gingivitis (no evidence of bone loss) and periodontal disease with evidence of bone loss cohorts compared to the healthy control cohort (p<0.01 and p<0.0001, respectively). FIG. 7 illustrates an oral microbiome based periodontal disease risk assessment in clinically recruited cohorts of cats suffering from gingivitis (no bone loss), periodontal disease and bone loss, and citizen science recruited healthy controls. The horizontal lines represent the mean risk score for each cohort (the risk score range is from 0 to 1, with higher values representing increased risk of disease) and the error bars represent the Standard Error of the Mean (SEM). A 2-tailed t-test assuming unequal variance was used for each comparison; *p<0.01, ***<0.0001.

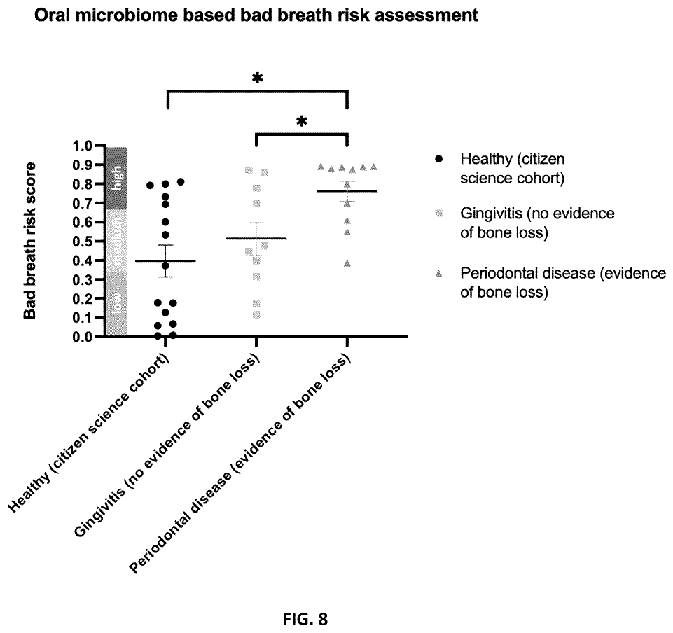

The algorithm generated bad breath (halitosis) risk assessment was significantly higher for the gingivitis (no evidence of bone loss) cohort compared to the periodontal disease with evidence of bone loss cohort (p<0.05). The halitosis risk assessment was also significantly higher for the periodontal disease with evidence of bone loss cohort compared to the healthy control cohort (p<0.01). Halitosis is a known hallmark of gingivitis and later stages of periodontal disease. FIG. 8 illustrates an oral microbiome based bad breath (halitosis) risk assessment in clinically recruited cohorts of cats suffering from gingivitis (no bone loss), periodontal disease and bone loss, and citizen science recruited healthy controls. The horizontal lines represent the mean risk score for each cohort (the risk score range is from 0 to 1, with higher values representing increased risk of disease) and the error bars represent the Standard Error of the Mean (SEM). A 2-tailed t-test assuming unequal variance was used for each comparison; *p<0.05.

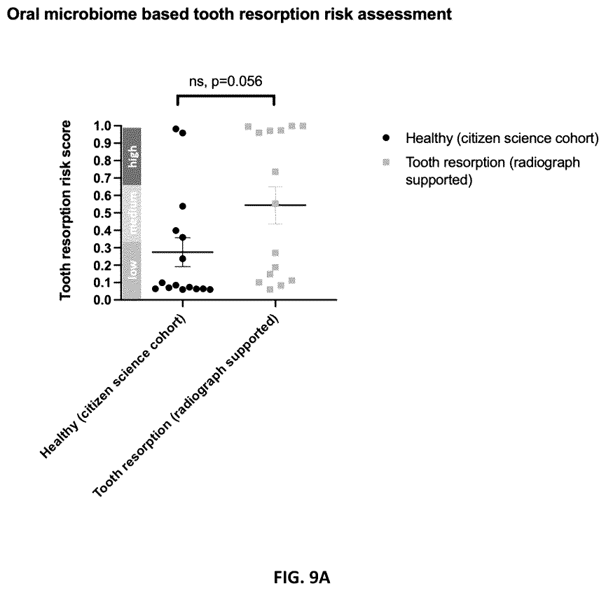

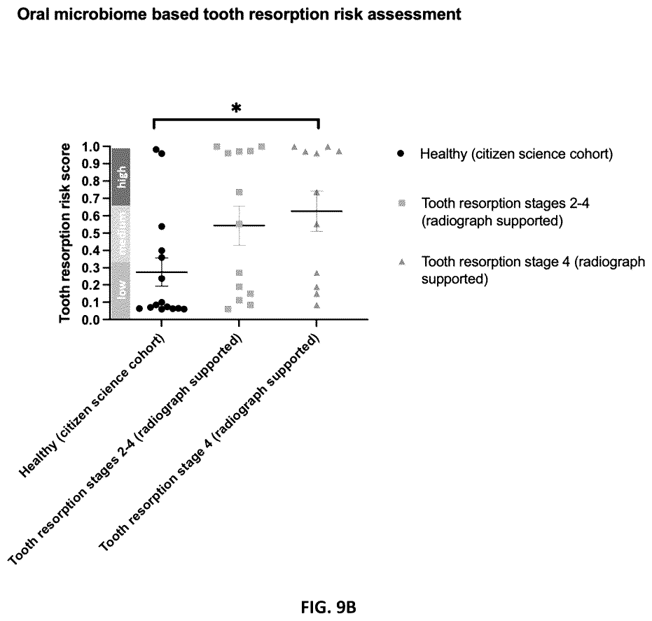

FIG. 9A illustrates an oral microbiome-based tooth resorption risk assessment in clinically recruited cohorts of cats suffering from tooth resorption and citizen science recruited healthy controls. FIG. 9B illustrates an oral microbiome-based tooth resorption risk assessment in clinically recruited cohorts of cats suffering from tooth resorption, incorporating tooth resorption staging information, and citizen science recruited healthy controls. There was a significant difference between the mean risk score generated for the healthy control cohort and the stage 4 tooth resorption cohort.

### Discussion

The disclosed methods' and systems' specificity and sensitivity are potentially influenced by the sample collection method. Current risk prediction models are based on pet owner-provided oral swab samples where the whole mouth was targeted for sample collection, focusing on no particular area of interest. As replicate studies have shown, risk assessments based on a ‘whole mouth’ swab sample can occasionally show variability (specific examples are Cat V's and Cat IX's samples from Study 2). This is probably due to the fact that when the pet owner is instructed to collect a ‘whole mouth’ swab sample, different mouth areas get preferentially swabbed each time. Since the easiest area to swab is the tongue, it is possible that some ‘whole mouth’ sample collection attempts focus on the tongue area where the presence of dental disease-associated microbes is more variable compared to the gum line. Interestingly, when there was a discrepancy between ‘whole mouth’ and ‘gum line’ targeted oral swab collection in Study 1, the ‘gum line’ risk score was always higher than the ‘whole mouth’ risk score (Cat #4, Cat #5, Cat #8, Cat #10). This supports the hypothesis that sample collection targeted at the gum line is likely to more accurately represent microbiome states linked to dental diseases.

For this reason, in Study 3, the veterinary technician was instructed to target the gum line for sample collection from cats suffering from periodontal disease and tooth resorption. The disclosed method was able to identify cats with early stages of periodontal disease (i.e., gingivitis with no evidence of alveolar bone loss) and cats with more advanced periodontal disease (with evidence of alveolar bone loss) as being at a significantly higher risk of periodontal disease than cats from the healthy citizen science-recruited cohort. Additionally, the disclosed method identified cats with periodontal disease and evidence of alveolar bone loss as being at a significantly higher risk of halitosis, compared to healthy controls. Similarly, cats with initial stages of periodontal disease (i.e., gingivitis with no evidence of alveolar bone loss) were found to be at a significantly higher risk of halitosis, compared to controls. Halitosis is commonly known to be a harbinger of periodontal disease. These results demonstrate the utility and accuracy of the disclosed methods and systems as a screening tool for cats at both early and late stages of periodontal disease.

Study 3 failed to demonstrate a significant difference in tooth resorption risk between cats with radiographic evidence of tooth resorption and the healthy citizen science-recruited cohort, unless the stage of tooth resorption was taken into account. The disclosed method identified cats with stage 4 tooth resorption as being at a significantly higher risk of the disease compared to healthy controls. As previously hypothesized, the disclosed method had the highest sensitivity when the resorptive lesion had reached the surface of the tooth and the tooth's integrity had already been compromised (stage 4). Stages 1 and 2 of tooth resorption are characterized with mild or moderate dental hard tissue loss that does not extend into the pulp cavity. In stage 3, the dentin loss extends into the pulp cavity, but most of the tooth still retains its integrity. Stage 5, on the other hand, is characterized by remnants of dental hard tissue and re-establishing of gingival covering over the affected area. The results of Study 3 indicate that the oral microbiome is most significantly altered as a consequence of tooth resorption at stage 4 (and leading to stage 5) of the disease.

It is already known that the colonization dynamics and influence of microorganisms (and their relative abundance) inhabiting organs and systems, such as the gastrointestinal tract (from mouth to anus), show many similarities between dogs (canines), cats (felines) and humans. Periodontal disease, which is a disease associated with microbial imbalance in the oral cavity, is prevalent among cats, dogs and humans, with some notable commonalities. The porphyromonas and treponema genera of bacteria, for example, play a critical role in periodontal disease pathogenesis in cats, dogs and humans.

The disclosed systems and methods use an oral swab collection device that has previously been successfully used for extracting genomic material from an oral swab sample from either cats or dogs. Additionally, per the manufacturer of the oral swab collection devices, the same swab collection device used is ideal for use with livestock (bovine, ovine, caprine), companion animals (canine, feline, equine) and other species by researchers, breeders, laboratories and consumers. The oral swab collection devices support use across different mammalian species. It has already been established that extraction of both host and microbial DNA from such sample collection devices is a possibility. It logically follows that the capability for this extraction on feline samples would extend to canine and other mammalian samples.

The disclosed systems and methods demonstrate analysis of the microbial species identity and abundance in feline oral microbiome samples for the purpose of screening for dental diseases in cats. Given that dogs and other mammals have oral cavities and oral microbiomes and are, in many cases, predisposed to the same dental disease pathologies (e.g., periodontal disease), our method should be readily applicable to dogs and other mammals. This is given that the model for each species is based on a comparison between a disease and a healthy animal cohort in order to derive the precise trends in microbial identities and abundances in each state for each species.

The degree to which the disclosed systems and methods enable detection, identification and indication of disease states such as periodontal disease, is further enabling for the detection, identification and indication of other disease states such as tooth resorption, feline gingivostomatitis and halitosis, among others. Similarly, the degree to which the disclosed systems and methods enable detection, identification and indication of disease states in felines, is further enabling for the detection, identification and indication of disease states in other mammalian animals, such as dogs (and other canines), horses (and other equines), sheep (and other ovines), cows (and other bovines and/or ruminates), pigs (and other porcine animals), guinea pigs, hamsters, etc.

The risk score generation methodology disclosed herein is based on oral microbiome compositional analysis. Other embodiments of the disclosed methods may also include incorporating predictions of the metabolic output of the oral microbiome (generated by enzymatic pathway analysis tools or metabolomics), alongside the oral microbiome compositional abundance analysis for the purpose of predictive risk of dental conditions.

### Additional Terms and Definitions

Unless defined otherwise, all technical and scientific terms used herein have the same meaning as commonly understood by one of ordinary skill in the art to which the present disclosure pertains.

Various “aspects” of the present disclosure, including systems, methods, and/or products may be illustrated with reference to one or more “embodiments,” which are exemplary in nature. As used herein, the terms “aspect” and “embodiment” may be used interchangeably. The term “embodiment” can also mean “serving as an example, instance, or illustration,” and should not necessarily be construed as preferred or advantageous over other aspects disclosed herein. In addition, reference to an “embodiment” of the present disclosure or invention is intended to provide an illustrative example without limiting the scope of the invention, which is indicated by the appended claims.

As used in this specification and the appended claims, the singular forms “a,” “an” and “the” each contemplate, include, and specifically disclose both the singular and plural referents, unless the context clearly dictates otherwise. For example, reference to a “protein” contemplates and specifically discloses one, as well as a plurality of (e.g., two or more, three or more, etc.) proteins. Similarly, use of a plural referent does not necessarily require a plurality of such referents, but contemplates, includes, specifically discloses, and/or provides support for a single, as well as a plurality of such referents, unless the context clearly dictates otherwise.

As used throughout this disclosure, the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

The term “condition” refers to any disorder, disease, injury, or illness, as understood by those skilled in the art, that is manifested or anticipated in a patient. Manifestation of such a condition can be an early, middle, or late stage manifestation, as known in the art, including pre-condition symptoms, signs, or markers. Anticipation of such a condition can be or include the predicted, expected, envisioned, presumed, supposed, and/or speculated occurrence of the same, whether founded in scientific or medical evidence, risk assessment, or mere apprehension or trepidation.

The term “patient,” as used herein, is synonymous with the term “subject” and generally refers to any animal under the care of a medical professional, as that term is defined herein, with particular reference to (i) humans (under the care of a doctor, nurse, or medical assistant or volunteer) and (ii) non-human animals, such as non-human mammals (under the care of a veterinarian or other veterinary professional, assistant, or volunteer).

“Mammal” includes humans and both domestic animals such as laboratory animals and household pets (e.g., cats, dogs, swine, cattle, sheep, goats, horses, rabbits), and non-domestic animals such as wildlife and the like.

“Treating” or “treatment” as used herein covers the treatment of the disease or condition of interest in a mammal, preferably a cat or dog, having the disease or condition of interest, and includes: (i) preventing the disease or condition from occurring in a mammal, in particular, when such mammal is actually starting to develop the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

For the sake of brevity, the present disclosure may recite a list or range of numerical values. It will be appreciated, however, that where such a list or range of numerical values (e.g., greater than, less than, up to, at least, and/or about a certain value, and/or between two recited values) is disclosed or recited, any specific value or range of values falling within the disclosed values or list or range of values is likewise specifically disclosed and contemplated herein.

To facilitate understanding, like references (i.e., like naming of components and/or elements) have been used, where possible, to designate like elements common to different embodiments of the present disclosure. Similarly, like components, or components with like functions, will be provided with similar reference designations, where possible. Specific language will be used herein to describe the exemplary embodiments. Nevertheless, it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

While the detailed description is separated into sections, the section headers and contents within each section are for organizational purposes only and are not intended to be self-contained descriptions and embodiments or to limit the scope of the description or the claims. Rather, the contents of each section within the detailed description are intended to be read and understood as a collective whole, where elements of one section may pertain to and/or inform other sections. Accordingly, embodiments specifically disclosed within one section may also relate to and/or serve as additional and/or alternative embodiments in another section having the same and/or similar products, methods, and/or terminology.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### TABLES

Table 1. Selected microbial species which show significantly increased or decreased compositional abundance in periodontal disease compared to control (p<0.05). The average percentage increased or decreased abundance for each microbial species when compared to a healthy control (calculated using a centered log-ratio transformation) is shown. Microbial species previously described in scientific literature as misregulated in periodontal disease are shown in bold font.

TABLE 1

| | % increase | | % decrease |
|---|---|---|---|
| Microbes with increased | compared to | Microbes with decreased | compared |
| abundance in PD cohort | Healthy cohort | abundance in PD cohort | Healthy cohort |
| Bacteroides sp. HF-5287 | +49% | Frederiksenia canicola | −47% |
| Bacteroides zoogleoformans | +47% | Moraxella bovis | −33% |
| Bacteroides sp. M10 | +41% | Mannheimia haemolytica | −32% |
| Odoribacter splanchnicus | +38% | Pseudoleptotrichia | −32% |
| | | goodfellowii | |
| Desulfobulbus oralis | +36% | Streptobacillus moniliformis | −32% |
| Bacteroides caccae | +35% | Capnocytophaga sp. H4358 | −29% |
| Desulfomicrobium orale | +35% | Capnocytophaga sp. H2931 | −29% |
| Bacteroides sp. CBA7301 | +33% | Moraxella catarrhalis | −28% |
| Bacteroides uniformis | +33% | Alysiella filiformis | −28% |
| Parabacteroides distasonis | +33% | Moraxella cuniculi | −27% |
| Bacteroides ovatus | +32% | Moraxella ovis | −27% |
| Bacteroides caecimuris | +32% | Moraxella bovoculi | −27% |
| Desulfovibrio fairfieldensis | +26% | Neisseria zoodegmatis | −27% |
| Porphyromonas gingivalis | +26% | Neisseria weaveri | −26% |
| Bacteroides heparinolyticus | +25% | Capnocytophaga | −25% |
| | | cynodegmi | |
| Actinomyces sp. Chiba101 | +25% | Neisseria animaloris | −25% |
| Bacteroides thetaiotaomicron | +25% | Cutibacterium acnes | −24% |
| Paraprevotella xylaniphila | +25% | Neisseria chenwenguii | −23% |
| Actinomyces howellii | +25% | Neisseria elongata | −22% |
| Bacteroides xylanisolvens | +24% | Neisseria dentiae | −22% |
| Bacteroides helcogenes | +24% | Kingella oralis | −22% |
| Petrimonas mucosa | +24% | Neisseria canis | −22% |
| Desulfovibrio desulfuricans | +24% | Pelistega sp. NLN63 | −21% |
| Bacteroides fragilis | +23% | Neisseria wadsworthii | −21% |
| Bacteroides sp. A1C1 | +22% | Moraxella osloensis | −21% |
| Treponema sp. OMZ 838 | +22% | Capnocytophaga | −21% |
| | | canimorsus | |
| Proteiniphilum | +22% | Epilithonimonas vandammei | −19% |
| saccharofermentans | | | |
| Treponema brennaborense | +22% | Lysobacter oculi | −19% |
| Treponema putidum | +22% | Streptococcus dysgalactiae | −18% |
| Treponema denticola | +21% | Riemerella anatipestifer | −18% |
| Treponema pedis | +11% | Capnocytophaga stomatis | −17% |
| Acidovorax monticola | +11% | Fusobacterium hwasookii | −17% |
| Propionibacterium freudenreichii | +11% | Cardiobacterium hominis | −17% |
| Treponema phagedenis | +11% | Acinetobacter johnsonii | −17% |
| Prevotella denticola | +10% | Neisseria shayeganii | −16% |
| Acidovorax sp. RAC01 | +10% | Fusobacterium | −16% |
| | | pseudoperiodonticum | |
| Tannerella forsythia | +10% | Pasteurella multocida | −16% |

TABLE 2
TABLE 2. Predictive microbes for periodontal disease, tooth resorption
and halitosis based on pairwise log-ratio microbial abundance
comparisons between healthy/control oral microbiomes and those
of cats suffering from one of the three dental conditions. Identification
of the predictive microbes is dynamic and will evolve as the
reference database and the cohorts evolve. ‘1’ indicates
that the microbe is considered predictive of a particular dental
condition, while ‘0’ indicates that it is not.

| | peri- | | |
|---|---|---|---|
| | odon- | resorp- | bad |
| Microbial species | tal | tion | breath |
| Haemophilus influenzae: 727 | 1 | 0 | 0 |
| Actinomyces howellii: 52771 | 1 | 0 | 0 |
| Saccharomyces cerevisiae: 4932 | 1 | 0 | 0 |
| Bacteroides intestinalis: 329854 | 1 | 0 | 0 |
| Bacteroides ovatus: 28116 | 1 | 0 | 0 |
| Tessaracoccus timonensis: 2161816 | 1 | 0 | 0 |
| Petrimonas mucosa: 1642646 | 1 | 0 | 0 |
| Porphyromonas asaccharolytica: 28123 | 1 | 0 | 0 |
| Prevotella scopos: 589437 | 1 | 0 | 0 |
| Acinetobacter guillouiae: 106649 | 1 | 0 | 0 |
| Corynebacterium tuberculostearicum: 38304 | 1 | 0 | 0 |
| Acinetobacter johnsonii: 40214 | 1 | 0 | 0 |
| Actinomyces sp. ZJ750: 2744574 | 1 | 0 | 0 |
| Kocuria indica: 1049583 | 1 | 0 | 0 |
| Bacteroides uniformis: 820 | 1 | 0 | 0 |
| Salmonella enterica: 28901 | 1 | 0 | 0 |
| Corynebacterium sp. ATCC 6931: 1487956 | 1 | 0 | 0 |
| Neisseria polysaccharea: 489 | 1 | 0 | 0 |
| Proteiniphilum saccharofermentans: 1642647 | 1 | 0 | 0 |
| Actinomyces sp. Chiba101: 1851395 | 1 | 0 | 0 |
| Odoribacter splanchnicus: 28118 | 1 | 0 | 0 |
| Flavonifractor plautii: 292800 | 0 | 1 | 0 |
| Tessaracoccus flavescens: 399497 | 0 | 1 | 0 |
| Psychrobacter sp. P11G5: 1699624 | 0 | 1 | 0 |
| Klebsiella grimontii: 2058152 | 0 | 1 | 0 |
| Escherichia coli: 562 | 0 | 1 | 0 |
| Ruthenibacterium lactatiformans: 1550024 | 0 | 1 | 0 |
| Streptococcus equi: 1336 | 0 | 1 | 0 |
| Enterocloster clostridioformis: 1531 | 0 | 1 | 0 |
| Parabacteroides distasonis: 823 | 0 | 1 | 0 |
| Lachnoanaerobaculum umeaense: 617123 | 0 | 1 | 0 |
| Aeromonas sp. ASNIH1: 1636606 | 0 | 1 | 0 |
| Acetoanaerobium sticklandii: 1511 | 0 | 1 | 0 |
| Histophilus somni: 731 | 1 | 1 | 0 |
| Alloprevotella sp. E39: 2133944 | 1 | 1 | 0 |
| Cruoricaptor ignavus: 1118202 | 1 | 1 | 0 |
| Pasteurella dagmatis: 754 | 1 | 1 | 0 |
| Psychrobacter alimentarius: 261164 | 1 | 1 | 0 |
| Fusobacterium pseudoperiodonticum: 2663009 | 1 | 1 | 0 |
| Fusobacterium gonidiaformans: 849 | 1 | 1 | 0 |
| Streptococcus dysgalactiae: 1334 | 1 | 1 | 0 |
| Streptococcus suis: 1307 | 1 | 1 | 0 |
| Dichelobacter nodosus: 870 | 1 | 1 | 0 |
| Acinetobacter junii: 40215 | 1 | 1 | 0 |
| Streptobacillus moniliformis: 34105 | 1 | 1 | 0 |
| Wolinella succinogenes: 844 | 1 | 1 | 0 |
| Moraxella bovoculi: 386891 | 1 | 1 | 0 |
| Streptococcus ruminantium: 1917441 | 1 | 1 | 0 |
| Saccharomyces eubayanus: 1080349 | 1 | 1 | 0 |
| Psychrobacter sp. PRwf-1: 349106 | 1 | 1 | 0 |
| Roseburia hominis: 301301 | 1 | 1 | 0 |
| Fusobacterium periodonticum: 860 | 1 | 1 | 0 |
| Corynebacterium xerosis: 1725 | 1 | 1 | 0 |
| Mycoplasma felis: 33923 | 1 | 1 | 0 |
| Leptotrichia sp. oral taxon 212: 712357 | 1 | 1 | 0 |
| Pelistega sp. NLN63: 2652177 | 1 | 1 | 0 |
| Streptococcus oralis: 1303 | 1 | 1 | 0 |
| Bacteroides xylanisolvens: 371601 | 1 | 1 | 0 |
| Moraxella osloensis: 34062 | 1 | 1 | 0 |
| Neisseria zoodegmatis: 326523 | 1 | 1 | 0 |
| Porphyromonas crevioricanis: 393921 | 0 | 0 | 1 |
| Prevotella jejuni: 1177574 | 0 | 0 | 1 |
| Campylobacter sp. RM16192: 1660080 | 0 | 0 | 1 |
| Serratia sp. FS14: 1327989 | 0 | 0 | 1 |
| Gemella morbillorum: 29391 | 0 | 0 | 1 |
| Schaalia odontolytica: 1660 | 0 | 0 | 1 |
| Acidovorax ebreus: 721785 | 0 | 0 | 1 |
| Luteimonas granuli: 1176533 | 0 | 0 | 1 |
| Citrobacter sp. RHBSTW-00599: 2742657 | 0 | 0 | 1 |
| Brucella intermedia: 94625 | 0 | 0 | 1 |
| Acidovorax sp. KKS102: 358220 | 0 | 0 | 1 |
| Treponema pedis: 409322 | 0 | 0 | 1 |
| Stenotrophomonas sp. SXG-1: 2682487 | 0 | 0 | 1 |
| Bordetella genomo sp. 6: 463024 | 0 | 0 | 1 |
| Streptococcus intermedius: 1338 | 0 | 0 | 1 |
| Diaphorobacter aerolatus: 1288495 | 0 | 0 | 1 |
| Filifactor alocis: 143361 | 0 | 0 | 1 |
| Pseudoxanthomonas spadix: 415229 | 0 | 0 | 1 |
| Citrobacter sp. RHBSTW-00570: 2742655 | 0 | 0 | 1 |
| Pseudomonas stutzeri: 316 | 0 | 0 | 1 |
| Arsenicicoccus sp. oral taxon 190: 1658671 | 0 | 0 | 1 |
| Thermomonas sp. HDW16: 2714945 | 0 | 0 | 1 |
| Treponema phagedenis: 162 | 0 | 0 | 1 |
| Acetobacter pasteurianus: 438 | 0 | 0 | 1 |
| Xanthomonas sacchari: 56458 | 0 | 0 | 1 |
| Bordetella parapertussis: 519 | 0 | 0 | 1 |
| Citrobacter sp. RHBSTW-00229: 2742641 | 0 | 0 | 1 |
| Xanthomonas hortorum: 56454 | 0 | 0 | 1 |
| Pseudoxanthomonas mexicana: 128785 | 0 | 0 | 1 |
| Xanthomonas euvesicatoria: 456327 | 0 | 0 | 1 |
| Pseudomonas otitidis: 319939 | 0 | 0 | 1 |
| Acidovorax sp. JS42: 232721 | 0 | 0 | 1 |
| Treponema sp. OMZ 838: 1539298 | 0 | 0 | 1 |
| Polaromonas sp. JS666: 296591 | 0 | 0 | 1 |
| Ralstonia mannitolilytica: 105219 | 0 | 0 | 1 |
| Thermomonas sp. XSG: 2771436 | 0 | 0 | 1 |
| Pseudomonas plecoglossicida: 70775 | 0 | 0 | 1 |
| Prevotella oris: 28135 | 0 | 0 | 1 |
| Variovorax boronicumulans: 436515 | 0 | 0 | 1 |
| Comamonas serinivorans: 1082851 | 0 | 0 | 1 |
| Enterobacter cloacae complex sp. FDA-CDC- | 0 | 0 | 1 |
| AR_0132: 2077137 | | | |
| Stenotrophomonas indicatrix: 2045451 | 0 | 0 | 1 |
| Micrococcus luteus: 1270 | 0 | 0 | 1 |
| Polaromonas naphthalenivorans: 216465 | 0 | 0 | 1 |
| Selenomonas sputigena: 69823 | 0 | 0 | 1 |
| Hydrogenophaga sp. NH-16: 2184519 | 0 | 0 | 1 |
| Acidovorax sp. T1: 1858609 | 0 | 0 | 1 |
| Treponema denticola: 158 | 0 | 0 | 1 |
| Ralstonia pseudosolanacearum: 1310165 | 0 | 0 | 1 |
| Citrobacter sp. RHB36-C18: 2742627 | 0 | 0 | 1 |
| Mycoplasma arginini: 2094 | 0 | 0 | 1 |
| Acidovorax carolinensis: 553814 | 0 | 0 | 1 |
| Pseudomonas denitrificans (nom. rej.): 43306 | 0 | 0 | 1 |
| Eubacterium callanderi: 53442 | 0 | 0 | 1 |
| Xanthomonas citri: 346 | 0 | 0 | 1 |
| Stenotrophomonas maltophilia: 40324 | 0 | 0 | 1 |
| Achromobacter ruhlandii: 72557 | 0 | 0 | 1 |
| Paracoccus yeei: 147645 | 0 | 0 | 1 |
| Hydrogenophaga sp. PBC: 795665 | 0 | 0 | 1 |
| Xanthomonas translucens: 343 | 0 | 0 | 1 |
| Xanthomonas arboricola: 56448 | 0 | 0 | 1 |
| Cutibacterium acnes: 1747 | 0 | 0 | 1 |
| Streptococcus pseudoporcinus: 361101 | 0 | 0 | 1 |
| Xanthomonas campestris: 339 | 0 | 0 | 1 |
| Campylobacter showae: 204 | 0 | 0 | 1 |
| Alcaligenes faecalis: 511 | 0 | 0 | 1 |
| Lysobacter enzymogenes: 69 | 0 | 0 | 1 |
| Acidovorax sp. 16-35-5: 2743470 | 0 | 0 | 1 |
| Streptococcus milleri: 33040 | 0 | 0 | 1 |
| Achromobacter xylosoxidans: 85698 | 0 | 0 | 1 |
| Prevotella intermedia: 28131 | 0 | 0 | 1 |
| Acidovorax sp. HDW3: 2714923 | 0 | 0 | 1 |
| Streptococcus anginosus: 1328 | 0 | 0 | 1 |
| Diaphorobacter sp. JS3050: 2735554 | 0 | 0 | 1 |
| Streptomyces sp. 2114.2: 1881022 | 0 | 0 | 1 |
| Aeromonas caviae: 648 | 0 | 0 | 1 |
| Comamonas sp. NLF-7-7: 2597701 | 0 | 0 | 1 |
| Citrobacter pasteurii: 1563222 | 0 | 0 | 1 |
| Enterobacter sp. DSM 30060: 2747372 | 0 | 0 | 1 |
| Bordetella hinzii: 103855 | 0 | 0 | 1 |
| Stenotrophomonas rhizophila: 216778 | 0 | 0 | 1 |
| Serpentinomonas raichei: 1458425 | 0 | 0 | 1 |
| Thermomonas brevis: 215691 | 0 | 0 | 1 |
| Enterobacter sp. RHBSTW-00593: 2742656 | 0 | 0 | 1 |
| Variovorax sp. HW608: 1034889 | 0 | 0 | 1 |
| Luteimonas sp. YGD11-2: 2508168 | 0 | 0 | 1 |
| Serpentinomonas mccroryi: 1458426 | 0 | 0 | 1 |
| Comamonas thiooxydans: 363952 | 0 | 0 | 1 |
| Lysobacter maris: 1605891 | 0 | 0 | 1 |
| Campylobacter rectus: 203 | 0 | 0 | 1 |
| Fusobacterium necrophorum: 859 | 0 | 0 | 1 |
| Bacteroides cellulosilyticus: 246787 | 0 | 0 | 1 |
| Serratia sp. LS-1: 2485839 | 0 | 0 | 1 |
| Aerosticca soli: 2010829 | 0 | 0 | 1 |
| Stenotrophomonas nitritireducens: 83617 | 0 | 0 | 1 |
| Pulveribacter suum: 2116657 | 0 | 0 | 1 |
| Delftia tsuruhatensis: 180282 | 0 | 0 | 1 |
| Phocaeicola dorei: 357276 | 0 | 0 | 1 |
| Achromobacter spanius: 217203 | 0 | 0 | 1 |
| Stenotrophomonas acidaminiphila: 128780 | 0 | 0 | 1 |
| Melaminivora sp. SC2-9: 2109913 | 0 | 0 | 1 |
| Citrobacter sp. CF971: 2566012 | 0 | 0 | 1 |
| Citrobacter freundii: 546 | 0 | 0 | 1 |
| Comamonas aquatica: 225991 | 0 | 0 | 1 |
| Hydrogenophaga sp. PBL-H3: 434010 | 0 | 0 | 1 |
| Propioniciclava sp. HDW11: 2714937 | 0 | 0 | 1 |
| Comamonas testosteroni: 285 | 0 | 0 | 1 |
| Treponema brennaborense: 81028 | 0 | 0 | 1 |
| Treponema putidum: 221027 | 0 | 0 | 1 |
| Alicycliphilus denitrificans: 179636 | 0 | 0 | 1 |
| Thauera hydrothermalis: 2184083 | 0 | 0 | 1 |
| Treponema socranskii: 53419 | 0 | 0 | 1 |
| Enterobacter roggenkampii: 1812935 | 0 | 0 | 1 |
| Proteus terrae: 1574161 | 0 | 0 | 1 |
| Bacteroides fragilis: 817 | 1 | 0 | 1 |
| Desulfobulbus oralis: 1986146 | 1 | 0 | 1 |
| Neisseria canis: 493 | 1 | 0 | 1 |
| Bacteroides caecimuris: 1796613 | 1 | 0 | 1 |
| Cardiobacterium hominis: 2718 | 1 | 0 | 1 |
| Lysobacter oculi: 2698682 | 1 | 0 | 1 |
| Prevotella denticola: 28129 | 1 | 0 | 1 |
| Pseudoleptotrichia goodfellowii: 157692 | 1 | 0 | 1 |
| Ottowia sp. oral taxon 894: 1658672 | 1 | 0 | 1 |
| Capnocytophaga sputigena: 1019 | 1 | 0 | 1 |
| Hydrogenophaga sp. BA0156: 2716225 | 1 | 0 | 1 |
| Tannerella forsythia: 28112 | 1 | 0 | 1 |
| Capnocytophaga cynodegmi: 28189 | 1 | 0 | 1 |
| Neisseria subflava: 28449 | 1 | 0 | 1 |
| Conchiformibius steedae: 153493 | 1 | 0 | 1 |
| Desulfovibrio sp. G11: 631220 | 1 | 0 | 1 |
| Desulfovibrio fairfieldensis: 44742 | 1 | 0 | 1 |
| Porphyromonas gingivalis: 837 | 1 | 0 | 1 |
| Actinomyces israelii: 1659 | 1 | 0 | 1 |
| Neisseria chenwenguii: 1853278 | 1 | 0 | 1 |
| Neisseria meningitidis: 487 | 1 | 0 | 1 |
| Neisseria mucosa: 488 | 1 | 0 | 1 |
| Neisseria elongata: 495 | 1 | 0 | 1 |
| Neisseria shayeganii: 607712 | 1 | 0 | 1 |
| Bacteroides heparinolyticus: 28113 | 1 | 0 | 1 |
| Neisseria bacilliformis: 267212 | 1 | 0 | 1 |
| Lautropia mirabilis: 47671 | 1 | 0 | 1 |
| Eikenella corrodens: 539 | 1 | 0 | 1 |
| Desulfomicrobium orale: 132132 | 1 | 0 | 1 |
| Bacteroides caccae: 47678 | 1 | 0 | 1 |
| Neisseria dentiae: 194197 | 1 | 0 | 1 |
| Alysiella filiformis: 194196 | 1 | 0 | 1 |
| Neisseria sicca: 490 | 1 | 0 | 1 |
| Streptococcus canis: 1329 | 0 | 1 | 1 |
| Gemella sp. oral taxon 928: 1785995 | 0 | 1 | 1 |
| Aerococcus sanguinicola: 119206 | 0 | 1 | 1 |
| Ottowia oryzae: 2109914 | 0 | 1 | 1 |
| Treponema sp. Marseille-Q4132: 2766701 | 0 | 1 | 1 |
| Parvimonas micra: 33033 | 0 | 1 | 1 |
| Treponema sp. OMZ 804: 120683 | 0 | 1 | 1 |
| Pseudopropionibacterium propionicum: 1750 | 0 | 1 | 1 |
| Bacteroides sp. HF-5287: 2650157 | 1 | 1 | 1 |
| Actinobacillus pleuropneumoniae: 715 | 1 | 1 | 1 |
| Capnocytophaga sp. H4358: 1945658 | 1 | 1 | 1 |
| Capnocytophaga canimorsus: 28188 | 1 | 1 | 1 |
| Rodentibacter pneumotropicus: 758 | 1 | 1 | 1 |
| Glaesserella sp. 15-184: 2030797 | 1 | 1 | 1 |
| Riemerella anatipestifer: 34085 | 1 | 1 | 1 |
| Bacteroides zoogleoformans: 28119 | 1 | 1 | 1 |
| Mannheimia sp. USDA-ARS-USMARC- | 1 | 1 | 1 |
| 1261: 1432056 | | | |
| Moraxella cuniculi: 34061 | 1 | 1 | 1 |
| Neisseria animaloris: 326522 | 1 | 1 | 1 |
| Haemophilus haemolyticus: 726 | 1 | 1 | 1 |
| Mannheimia pernigra: 111844 | 1 | 1 | 1 |
| Neisseria weaveri: 28091 | 1 | 1 | 1 |
| Frederiksenia canicola: 123824 | 1 | 1 | 1 |
| Pasteurella multocida: 747 | 1 | 1 | 1 |
| Capnocytophaga sp. H2931: 1945657 | 1 | 1 | 1 |
| Neisseria musculi: 1815583 | 1 | 1 | 1 |
| Avibacterium paragallinarum: 728 | 1 | 1 | 1 |
| Actinobacillus indolicus: 51049 | 1 | 1 | 1 |
| Glaesserella parasuis: 738 | 1 | 1 | 1 |
| Aggregatibacter aphrophilus: 732 | 1 | 1 | 1 |
| Moraxella catarrhalis: 480 | 1 | 1 | 1 |
| Capnocytophaga stomatis: 1848904 | 1 | 1 | 1 |
| Moraxella ovis: 29433 | 1 | 1 | 1 |
| Neisseria wadsworthii: 607711 | 1 | 1 | 1 |
| Rodentibacter heylii: 1906744 | 1 | 1 | 1 |
| TOTAL | 108 | 74 | 182 |

TABLE 3
TABLE 3. Demographic statistics for
the cohorts of felines in Study 3.

| | Average | Gender |
|---|---|---|
| Cohort | age | split (M/F) |
| Gingivitis (no radiographic evidence of bone loss) | 6 years | 2/8 |
| Periodontal disease (radiographic evidence of bone | 9 years | 6/5 |
| loss) | | |
| Tooth resorption (with radiographic evidence) | 9 years | 7/8 |
| Healthy - owner provided information | 4 years | 10/5 |

### CONCLUSION

While the foregoing detailed description makes reference to specific exemplary embodiments, the present disclosure may be embodied in other specific forms without departing from its spirit or essential characteristics. Accordingly, the described embodiments are to be considered in all respects only as illustrative and not restrictive. For instance, various substitutions, alterations, and/or modifications of the inventive features described and/or illustrated herein, and additional applications of the principles described and/or illustrated herein, which would occur to one skilled in the relevant art and having possession of this disclosure, can be made to the described and/or illustrated embodiments without departing from the spirit and scope of the disclosure as defined by the appended claims. Such substitutions, alterations, and/or modifications are to be considered within the scope of this disclosure.

The scope of the invention is, therefore, indicated by the appended claims rather than by the foregoing description. The limitations recited in the claims are to be interpreted broadly based on the language employed in the claims and not limited to specific examples described in the foregoing detailed description, which examples are to be construed as non-exclusive and non-exhaustive. All changes which come within the meaning and range of equivalency of the claims are to be embraced within their scope.

It will also be appreciated that various features of certain embodiments can be compatible with, combined with, included in, and/or incorporated into other embodiments of the present disclosure. For instance, systems, methods, and/or products according to certain embodiments of the present disclosure may include, incorporate, or otherwise comprise features described in other embodiments disclosed and/or described herein. Thus, disclosure of certain features relative to a specific embodiment of the present disclosure should not be construed as limiting application or inclusion of said features to the specific embodiment.

In addition, unless a feature is described as being requiring in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. It will be appreciated that while features may be optional in certain embodiments, when features are included in such embodiments, they can be required to have a specific configuration as described in the present disclosure.

Likewise, any steps recited in any method or process described herein and/or recited in the claims can be executed in any suitable order and are not necessarily limited to the order described and/or recited, unless otherwise stated (explicitly or implicitly). Such steps can, however, also be required to be performed in a specific order or any suitable order in certain embodiments of the present disclosure.

Furthermore, various well-known aspects of illustrative systems, methods, products, and the like are not described herein in particular detail in order to avoid obscuring aspects of the example embodiments. Such aspects are, however, also contemplated herein.

1. A method for screening for, detecting, and/or preventing oral disease in non-human, mammalian animals, the method comprising:
obtaining an oral microbial profile for a non-human, mammalian animal, the oral microbial profile comprising one or more microbial species present in an oral sample of the non-human, mammalian animal and a quantity or abundance of the one or more microbial species in the oral sample;
comparing the oral microbial profile to information in a database that identifies weighted correlations between:
(i) occurrence and/or prevalence of one or more oral diseases in animals in a classification of the non-human, mammalian animal; and
(ii) presence and/or abundance of various microbial species in the oral microbiome of animals in the classification of the non-human, mammalian animal, wherein the various microbial species comprise the one or more microbial species in the oral sample;
generating a risk score indicating a likelihood that the non-human, mammalian animal has the one or more oral diseases based on one or more matches between the oral microbial profile and the information in the database; and
categorizing the non-human, mammalian animal as developing the one or more oral diseases when the risk score meets or exceeds a predetermined threshold and, optionally, prescribing a therapeutic treatment protocol suitable for treating, mitigating, or preventing the development, advancement, or recurrence of the one or more oral diseases when the risk score meets or exceeds a predetermined threshold.

2. The method of claim 1 further comprising administering the therapeutic treatment protocol to the non-human, mammalian animal or confirming that the therapeutic treatment protocol has been administered to the non-human, mammalian animal, wherein the therapeutic treatment protocol is sufficient to alter the oral microbial profile of the non-human, mammalian animal.

3. The method of claim 1, wherein obtaining the oral microbial profile for the non-human, mammalian animal comprises:
obtaining nucleic acid sequence data corresponding to microbial nucleic acid obtained from the oral sample;
analyzing the nucleic acid sequence data to identify the one or more microbial species present in the oral sample and quantifying the one or more microbial species; and
generating the oral microbial profile for the non-human, mammalian animal based on the identified and, optionally, quantified one or more microbial species.

4. The method of claim 3, wherein obtaining the microbial nucleic acid sequence data comprises:
sequencing microbial nucleic acid from the oral sample; and, optionally,
isolating the microbial nucleic acid from the oral sample.

5. The method of claim 4, wherein isolating the microbial nucleic acid from the oral sample comprises:
performing heat treatment on the oral sample; and
performing magnetic SPRI beads-based nucleic acid extraction on the heat-treated oral sample, with or without the addition of protein digesting reagents and detergents, to extract the microbial nucleic acid from the oral sample.

6. The method of claim 3, wherein analyzing the microbial nucleic acid sequence data comprises one or more of:
demultiplexing the nucleic acid sequence data;
trimming the nucleic acid sequence data;
mapping one or more unmapped reads onto a reference genome of the non-human, mammalian animal and/or onto existing microbial reference genomes;
classifying one or more reads as mammalian from the nucleic acid sequence data after mapping;
classifying one or more reads as microbial from the nucleic acid sequence data after mapping;
quantifying the one or more microbial reads;
transforming the quantified one or more microbial reads to account for sequence coverage biases using methods such as pairwise log ratio transformation; and
comparing compositional abundance patterns in the transformed one or more microbial reads against compositional abundance patterns in the transformed data in a reference database comprising samples from non-human, mammalian animals that do not suffer from dental diseases, as well as samples from non-human, mammalian animals that suffer from specific dental diseases.

7. The method of claim 1, wherein comparing the oral microbial profile to the information in the database comprises one or more of:
calculating the abundance of the one or more microbial species in the oral sample;
identifying the one or more microbial species in the oral sample; and
comparing the abundance of the identified one or more microbial species in the oral sample to the presence and/or abundance of various microbial species in the oral microbiome of animals in the classification of the non-human, mammalian animal contained in the database.

8. The method of claim 1, wherein generating the risk score comprises one or more of:
identifying one or more similarities between compositional abundance of the one or more microbial species in the oral sample and compositional abundance of various microbial species in the oral microbiome of animals in the classification of the non-human, mammalian animal contained in the database;
identifying one or more matches between the identity of the one or more microbial species in the oral sample and the presence of various microbial species in the oral microbiome of animals in the classification of the non-human, mammalian animal contained in the database;
quantifying the identified one or more similarities between the compositional abundance of the one or more microbial species in the oral sample and the compositional abundance of the one or more microbial species in the oral microbiome of animals in the classification of the non-human, mammalian animal contained in the database; and
identifying a presence of one or more predictive microbial species in the oral sample.

9. The method of claim 1, wherein the one or more oral diseases is selected from the group consisting of periodontal disease, tooth resorption, gingivostomatitis, and halitosis.

10. The method of claim 1 further comprising:
generating a report presenting (i) the risk score, (ii) an indication of developing the one or more oral diseases when the risk score meets or exceeds the predetermined threshold, (iii) a timing recommendation, (iv) optionally, one or more at home practices to improve dental health, (v) optionally, one or more diagnostic steps to diagnose the one or more oral diseases when the risk score meets or exceeds the predetermined threshold, and (vi) optionally, a prescription for the therapeutic treatment protocol; and, optionally,
communicating the generated report electronically to an owner of the non-human, mammalian animal and/or their veterinarian.

11. The method of claim 1, wherein the therapeutic treatment protocol is sufficient to alter the oral microbial profile of the non-human, mammalian animal.

12. A computer system configured to indicate or predict oral disease in mammalian animals, the computer system comprising:
one or more processors; and
one or more computer-readable hardware storage devices having stored thereon instructions that are executable by the one or more processors to configure the computer system to:
receive microbial nucleic acid sequence data corresponding to microbial nucleic acid obtained from an oral sample taken from a mammalian animal;
analyze the microbial nucleic acid sequence data to identify one or more microbial species present in the oral sample and quantify the one or more microbial species;
generate an oral microbial profile for the mammalian animal based on the identified one or more microbial species and their respective abundances;
compare the oral microbial profile to information in a database that identifies weighted correlations between:
(i) occurrence and/or prevalence of one or more oral diseases in animals in a classification of the mammalian animal; and
(ii) presence and/or abundance of various microbial species in the oral microbiome of animals in the classification of the mammalian animal, wherein the various microbial species comprise the one or more microbial species in the oral sample;
identify one or more matches between the oral microbial profile and the information in the database;
generate a risk score indicating a likelihood that the mammalian animal has the one or more oral diseases based on the one or more matches between the oral microbial profile and the information in the database; and, optionally,
diagnose the mammalian animal as “developing” the one or more oral diseases when the risk score meets or exceeds a predetermined threshold,
prescribe a therapeutic treatment protocol suitable for treating or preventing the one or more oral diseases when the risk score meets or exceeds the predetermined threshold,
generate a report indicating (i) the risk score, (ii) an indication of developing the one or more oral diseases when the risk score meets or exceeds the predetermined threshold, (iii) a timing recommendation, (iv) optionally, one or more at home practices to improve dental health, (v) optionally, one or more diagnostic steps to diagnose the one or more oral diseases when the risk score meets or exceeds the predetermined threshold, and (vi) a prescription for the therapeutic treatment protocol, and/or
communicate the generated report electronically to an owner of the mammalian animal and/or their veterinarian.

13. The computer system of claim 12, wherein the instructions further configure the computer system to map one or more unmapped reads to a mammalian reference genome and/or map one or more reads to microbial reference genomes and, optionally, classify the reads as microbial or mammalian.

14. The computer system of claim 13, wherein the instructions further configure the computer system to identify at least one unmapped sequence read of the metagenomic sequence data and, optionally, classify the at least one unmapped read.

15. The computer system of claim 13, wherein mammalian oral microbiome samples having fewer than 10,000 classified microbial reads or more than 500,000 classified microbial reads are excluded from the comparison of the oral microbial profile for the mammalian animal against a database of defined microbial profiles.

16. The computer system of claim 12, wherein the instructions further configure the computer system to calculate an abundance of the one or more microbial species present in the oral sample.

17. The computer system of claim 16, wherein the abundance of the specific one or more microbial species present in the oral sample correlates to whether the specific one or more microbial species is a predictive microbial species for the specific oral disease.

18. The computer system of claim 16, wherein the instructions further configure the computer system to perform a pairwise log ratio comparison of the microbial abundance of the mammalian animal's oral sample against the information in the database.

19. The system of claim 18, wherein the specific one or more microbial species is a predictive microbial species when 50% or more of the maximum possible pairwise log ratio comparisons involving this microbe are significantly different when compared between a disease and a control cohort.

20. A method for predicting the development of an oral disease in a mammalian animal, the method comprising:
obtaining an oral sample from a mammalian animal, the oral sample containing one or more microbial species;
isolating, from the oral sample, microbial nucleic acid of the one or more microbial species;
obtaining microbial nucleic acid sequence data corresponding to the microbial nucleic acid;
analyzing the microbial nucleic acid sequence data to identify one or more microbial species present in the oral sample and, optionally, quantifying the one or more microbial species;
generating an oral microbial profile for the mammalian animal based on the identified and, optionally, quantified one or more microbial species, the oral microbial profile comprising the one or more microbial species and, optionally, a quantity or relative abundance of the one or more microbial species in the oral sample;
comparing the oral microbial profile to information in a database that identifies weighted correlations between:
(i) occurrence and/or prevalence of one or more oral diseases in animals in a classification of the mammalian animal; and
(ii) presence and/or abundance of various microbial species in the oral microbiome of animals in the classification of the mammalian animal, wherein the various microbial species comprise the one or more microbial species in the oral sample;
generating a risk score indicating a likelihood of the mammalian animal developing the one or more oral diseases based on one or more matches between the oral microbial profile and the information in the database; and
indicating the mammalian animal as developing the one or more oral diseases when the risk score meets or exceeds a predetermined threshold.
